(19) United States
(12) Patent Application Publication
(10) Pub. No.: US 2021/0121835 A1
(43) Pub. Date: Apr. 29, 2021

(54) NANO EMULSION PROCESS FOR SCENTED LIQUIDS

(71) Applicant: OVR Tech, LLC, Burlington, VT (US)

(72) Inventors: Aaron Wisniewski, Burlington, VT (US); Sarah Socia, Burlington, VT (US); Matthew Flego, Burlington, VT (US); Samuel Wisniewski, Burlington, VT (US); Erik Cooper, Burlington, VT (US)

(73) Assignee: OVR Tech, LLC, Burlington, VT (US)

(21) Appl. No.: 17/031,841

(22) Filed: Sep. 24, 2020

Related U.S. Application Data
(60) Provisional application No. 62/905,851, filed on Sep. 25, 2019.

(51) Int. Cl. B01F 3/08; A61L 9/012

(52) U.S. Cl. CPC B01F 3/0819; B82Y 40/00; A61L 9/012

(57) ABSTRACT

Methods and devices for formulating scented nanoemulsions and dispersing one or more scents is disclosed. In some embodiments, the method includes providing a first mixture including water and a water surfactant, providing a second mixture including a fragrance material and an fragrance surfactant, mixing the first and second mixtures to create a temporary emulsion, and performing one or more high-energy homogenizations to the temporary emulsion until one or more desired physical properties of a resulting nanoemulsion are obtained. In some embodiments, the one or more high-energy homogenizations includes microfludization, sonication, and high-shear mixing. In some embodiments, the resulting nanoemulsion may thereafter be dispersed as a scent via an aerosolizing device. In some embodiments, the aerosolizing device may disburse scents in response to actions and/or events experienced in an AV/AR system.

### RELATED APPLICATIONS

This application claims the benefit under 35 U.S.C. § 119(e) to U.S. Application Ser. No. 62,905,851, entitled NANO EMULSION PROCESS FOR SCENTED LIQUIDS” and filed on Sep. 25, 2019, which is hereby incorporated by reference in its entirety.

### BACKGROUND

There are many methods for producing fragrances, including ones used in a variety of environments and systems. Some are passive, such as those with degrading media like those in household air fresheners. Others are sophisticated systems using active devices that control the release of scented media into the air.

### SUMMARY

According to some embodiments, a method of formulating scented nanoemulsions includes providing a first mixture including water and a water surfactant, providing a second mixture including a fragrance material and a fragrance surfactant, mixing the first and second mixtures to create a temporary emulsion, and performing one or more high-energy homogenizations to the temporary emulsion until one or more desired physical properties of a resulting nanoemulsion are obtained.

Still other aspects, examples, and advantages of these exemplary aspects and examples, are discussed in detail below, in conjunction with the accompanying drawings. Moreover, it is to be understood that both the foregoing information and the following detailed description are merely illustrative examples of various aspects and examples, and are intended to provide an overview or framework for understanding the nature and character of the claimed aspects and examples. Any example disclosed herein may be combined with any other example in any manner consistent with at least one of the objects, aims, and needs disclosed herein, and references to “an example,” “some examples,” “an alternate example,” “various examples,” “one example,” “at least one example,” “ this and other examples” or the like are not necessarily mutually exclusive and are intended to indicate that a particular feature, structure, or characteristic described in connection with the example may be included in at least one example. The appearances of such terms herein are not necessarily all referring to the same example.

### BRIEF DESCRIPTION OF DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. In the figures:

FIG. 1A illustrates a process for processing a liquid that may be used with one or more devices disclosed herein;

FIG. 1B illustrates another process for processing a liquid that may be used with one or more devices disclosed herein;

FIG. 2 is a schematic representation of a system used to process a liquid, such as via the processes shown in FIGS. 1A and 1B;

FIG. 3 shows a block diagram of a distributed computer system capable of implementing various aspects of the present disclosure;

FIG. 4 shows an example olfactory stimulus system according to some embodiments;

FIG. 5 shows another example olfactory stimulus system according to some embodiments;

FIG. 6 shows an example olfactory stimulus system physical configuration according to various embodiments;

FIG. 7 shows an example control system according to various embodiments;

FIG. 8 shows example scent classification information that may be used according to various aspects;

FIG. 9 shows an example process for rendering scent information according to various aspects;

FIGS. 10A-10B show example data formats for communicating scent information according to various embodiments;

FIG. 11 shows an example software architecture according to some embodiments;

FIG. 12 shows an example device that may be used to render scent according to some embodiments;

FIG. 13 shows an example device that may use one or more devices to render various scents according to some embodiments;

FIG. 14 shows another device that many be used to render various scents according to some embodiments;

FIGS. 15A-15D show a device for generating atomized fluid according to some embodiments;

FIG. 16 shows an alternative control system according to some embodiments;

FIG. 17 shows another alternative control system according to some embodiments;

FIGS. 18A-18E show various views of an example olfactory stimulus system according to some embodiments;

FIGS. 19A-19B show a device for generating atomized fluid according to some embodiments; and

FIG. 20 shows a more detailed view of an element including a tube assembly according to some embodiments.

### DETAILED DESCRIPTION

Historically, most scent formulations are solvent or oil-based formulas and are intended for use as a fragrance or for household care. It is appreciated that the physical properties of solvent and oil-based formulas are limiting to their applications due to their surface tension and viscosity. Also, the safety of solvent based formulas in close proximity to skin, lungs and membranes is also questionable.

In some embodiments, the inventors have appreciated that an improved process may be provided for producing scented liquids capable of being used with an aerosol generator. In some embodiments, various properties of the liquid are adjustable to improve the performance of the aerosol generator. For example, the inventors have appreciated that a scented nanoemulsion may be produced that includes one or more parameters that may be adjusted for use in an aerosol-producing device, such as those described by way of example in the exemplary systems described herein. In some embodiments, the inventors have appreciated that some parameters of the scented liquid may be adjusted such that the scented liquid has physical properties close to that of water, and that advantages may be realized by such a scented liquid.

For example, in some embodiments described herein, the viscosity of the liquid may be maintained to be between about 1 Cp and about 24 Cp at 20° C. For example, in some embodiments, the viscosity may between about 1 Cp and about 12 Cp. In some embodiments, the particle size may be maintained to be between about 1 nm and about 5000 nm. For example, the particle (e.g., droplet) size of the liquid may be maintained to be between about 1 nm and about 150 nm. In some embodiments, the surface tension of the liquid may be maintained at between about 20 mN/m and about 72 mN/m at 20° C. For example, the surface tension may be between 48 mN/m and about 72 mN/m. One or more of these parameters may be combined to optimize aerosol generation within a scent producing device. Although it should be appreciated that certain ranges or values may be used for particular parameters, it should be appreciated that these parameters may be adjusted differently, and certain embodiments are not limited to the ranges or values described herein.

Further, it should be appreciated that other aspects of the liquid may be adjusted, such as processing the liquid to be substantially particulate free prior to the aerosol generation step. Further, the liquid may be processed so as to be kinetically stable.

In some embodiments, a process is disclosed for processing a liquid that may be used, for example, in an aerosol generation device that produces scent for one or more application. For example, the process may include one or more steps for combining water and a fragrance material into a liquid soluable for use by an aerosol generator.

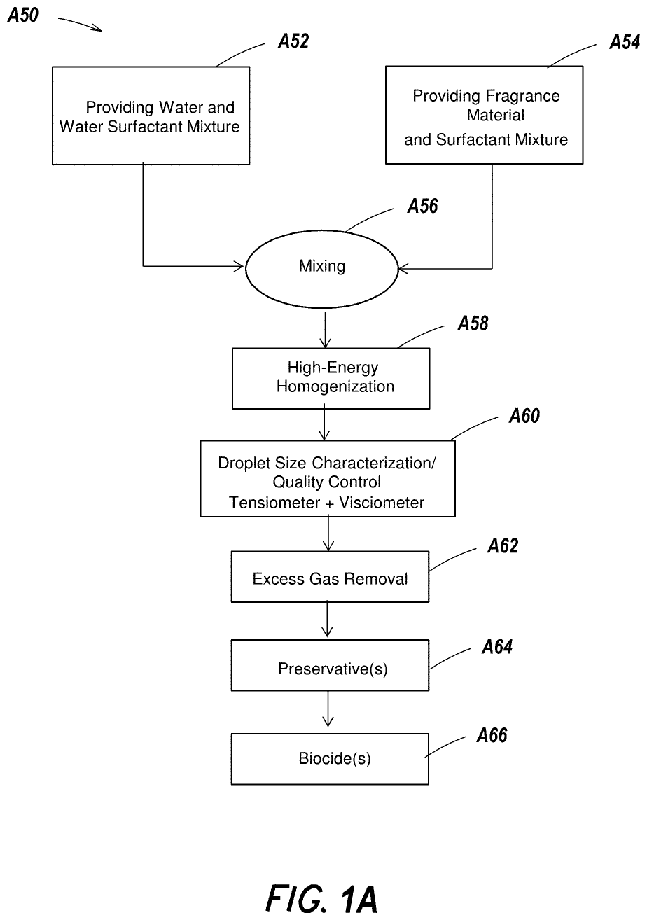

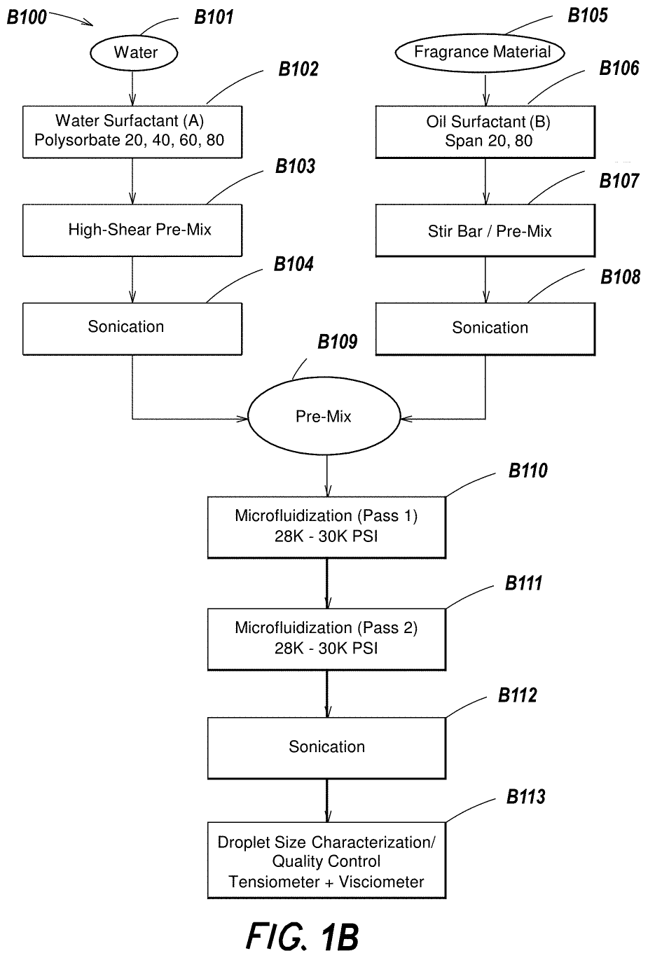

FIG. 1A illustrates one process A50 for processing the liquid for use, such as in the aerosol generation device. In some embodiments, as shown in this figure, the method includes providing a first mixture with water and a water surfactant A52, providing a second mixture with a fragrance material and a surfactant (e.g., an oil surfactant) A54, and mixing the first and second mixtures (e.g., the water phase and the oil phase) to form a temporary emulsion A56. In some embodiments, the step of providing the first mixture may include mixing the water and water surfactant (e.g., via a stir bar). Providing the first mixture also may include performing one or more additional processes on the water and water surfactant mixture (e.g., a high-energy homogenization). As with the water and water surfactant mixture, in some embodiments, providing the fragrance material and surfactant mixture also may include mixing and/or a high-energy homogenization.

Next, as shown at block A58, one or more high-energy homogenization processes (e.g., via a microfluidizer, an ultrasonic homogenizer, and/or a high-sheer rotor-stator) may be performed to the temporary emulsion until the desired physical characteristics are obtained for the resulting nanoemulsion. At block A60, a quality control step may be performed to confirm the characteristics of the nanoemulsion. For example, the nanoemulsion may have the above-noted particle size, viscosity, and/or surface tension.

Next, the nanoemulsion may be processed to remove excess gasses (see block A62), one or more preservatives may be added (see block A64), and/or one or more biocides may be added (see block A64). In some embodiments, removal of excess gas, addition of preservatives, and/or addition of biocides may allow the nanoemulsion to remain shelf stable. In some embodiments, as will be appreciated, after the quality control step of A60, the nanoemulsioin may be usable with the aerosol generation device.

FIG. 1B shows another process B100 for processing the liquid for use, such as in the aerosol generation device. As shown in this view, process B100 may include one or more steps for combining water (e.g., water B101) with a fragrance material (e.g., material B105) into a liquid suitable for use by the aerosol generator. In some embodiments (see block B102), water may be combined with the water surfactant, such as for example, polysorbate 20, 40, 60, 80, or another water surfactant. The resulting mixture may then be premixed using a high-shear mixing process (e.g., a high-energy homogenization process) at block B103, such as can be performed by a high-shear mixing blade rotating at a high rate of rotation. In some embodiments, such premixing may be performed via a micro rotor. As will be appreciated, the water and water surfactant may be mixed via other suitable methods in other embodiments (e.g., a stir bar). In some embodiments, the resulting mixture may then be subjected to a sonication process B104. As is known, sonication may include acts of applying sound energy to agitate particles in a sample for various purposes. Sonication may be used for the production of nanoparticles such as nanoemulsions, which are mixtures of two or more liquids that are normally unmixable (e.g., oil-based and water-based materials). In some embodiments, water and fragrance material may be combined with surfactants and subjected to respective sonication processes and then mixed together.

As shown in FIG. 1B, fragrance material (e.g., material B105) may be combined with an oil surfactant, such as span 20, 80, or another oil-based surfactant type. The resulting mixture may be premixed with, for example, a stir bar at block B107, although other mixing processes may be used. As shown at block B108, the mixture also may be subjected to a sonication process, such as that described above.

The resulting mixtures may then be combined to prepare an emulsion (e.g., a temporary emulsion). For example, at block B109, the mixtures are subjected to a second-level premixing process. In some embodiments, this second-level mixing process also may be a high-shear mixing process (e.g., with a high-sheer rotor stator). The resulting mixture may then be subjected to additional high-energy homogenization processes, such as a microfluidization process at block B110. For example, microfluidization at a pressure range of 28K-30K psi may be performed. As will be appreciated other high-energy homogenization processes (e.g., via an ultrasonic homogenizer and/or a high-shear rotor-stator) may be performed in other embodiments at block B110. Optionally, as shown at block B111, the resulting mixture may be subjected to additional microfluidization processes in further passes with the same or varying pressures. The resulting emulsion may then be subjected to a further sonication process at block B112.

In some embodiments, at block A113, the resulting emulsion mixture may be subjected to a quality control step where characteristics of the resulting emulsion are measured. For example, in some embodiments, droplet size, surface tension, and/or viscosity of the resulting mixture may be measured. As will be appreciated, other characteristics may be measured in other embodiments. As will be further appreciated, certain properties may be measured using one or more devices as know, such as, a tensiometer and viscometer. In some embodiments, if the resulting emulsion is found to be acceptable, the resulting nanoemulsion may be used within an aerosol generator (examples described below) for generating one or more scents. In some embodiments, if the resulting emulsion is not found to be acceptable, additional high-energy homogenization processes may be performed, and the resulting nanoemulsion may be retested at block B113.

In some embodiments, an acceptable resulting nanoemulsion may have a viscosity that is measured and maintained to be between about 1 Cp and about 24 Cp (at 20° C.), such as between about 1 Cp and about 12 Cp. The acceptable nanoemulsion also may have a droplet size that is between about 1 nm and about 5000 nm, such as between about 1 nm and about 150 nm, depending on the aerosol generator used. Further, in some embodiments, the surface tension of the acceptable resulting nanoformulation may be maintained to have a value of between about 20 mN/m and about 72 mN/m, such as between 48 mN/m and about 72 mN/m. It should be appreciated, however, that some of these parameters may be adjusted based on the generator and application.

As will be appreciated, after the quality control step at block B113, the nanoemulsion may be further processed, such as the nanoemulsion in FIG. 1A, to remove excess gasses, to add preservatives and/or to add biocides.

In some embodiments, the fragrance material may include hydrophobic molecules, such as oils, waxes, and/or powders. In such embodiments, the oils may include essential oils and/or synthetic oils. In some embodiments, the fragrance material may include a lipid-based material and/or a hydrophobic material. In some embodiments, the lipid-based material may be dissolved and/or readily dissolvable in water. In some embodiments, an oleo resin and/or a concrete material may be used. In some embodiments, the surfactant may include an emulsifier.

In an exemplary scent, an orange scent, the resulting material may include about 5% by weight of an orange essential oil, about 1% by weight of a surfactant, and the balance (e.g., about 94% by weight) of water. In another exemplary scent, a grass scent, the resulting mixture may include about 1% by weight of cis-3-hexanol, about 0.5% by weight of a surfactant, and the balance of water. In still another exemplary scent, a citrus scent, the resulting mixture may include about 2% by weight of a lemon essential oil, about 2% by weight of a lime essential oil, about 1% by weight of a grapefruit essential oil, about 1% by weight of a surfactant, and the balance in water.

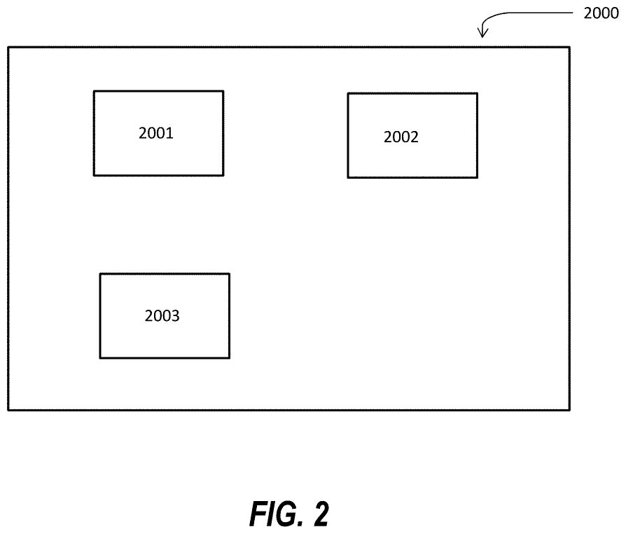

In some embodiments, the above-described processes A50, B100 may be performed via a system 2000 having one or more stations arranged to complete one or more process steps. For example, as shown in FIG. 2, the system may include a first station for mixing 2001, a second station for high-energy homogenization 2002, and a third station for quality control testing 2003.

In some embodiments, each station may be used multiple times to complete a process shown in FIGS. 1A and 1B. For example, a user may use the mixing station to mix the fragrance material and surfactant (see, e.g., blocks A54 and B107) and/or to mix the resulting mixtures (see, e.g., block A56) to create the temporary emulsion. In such an example, the mixing station may include a magnetic stirrer that may be used with a magnetic stir bar, or other suitable mixing devices. The mixing station also may be used to add the preservatives and/or biocides to the nanoemulsion in some embodiments, although these processes also may be performed at another, separate station.

As another example, the high-energy homogenization station 1002 may be used to perform sonication of the mixed water and water surfactant mixture (see, e.g., block B104), sonication of the mixed fragrance and oil surfactant mixture (see, e.g., block B108), and microfluidization of the resulting mixtures (see, e.g., blocks B110 and B111). In such an example, as will be appreciated, the high-energy homogenization station may include a microfluidizer, an ultrasonic homogenizer, and/or a high-shear rotor-stator.

Although a single station is shown for performing the same type of process steps, the system may include multiple stations that each perform the same process step(s). For example, in some embodiments, the system may include a first station for performing microfluidizations and a second station for performing sonications. In another example, the system may include two mixing stations, one used solely for mixing the fragrance material and oil surfactant, and a second for mixing only the water and fragrances.

In other embodiments, the same station may be arranged to perform different process steps. For example, in some embodiments, a first station may be arranged to perform the mixing and high-energy homogenization of the water and water surfactant (see, e.g., blocks B102-B104), a second station may be arranged to perform the mixing of the fragrance material and oil surfactant and the later sonication of the mixture (see, e.g., blocks B 106-B108), and a third station may be arranged to mix the resulting first and second mixtures and perform one or more high-energy homogenization steps (e.g., microfluidization and sonication at blocks B109-B112).

In still another embodiment, the system may include a single device that is capable of performing all of the process steps shown in FIGS. 1A and 1B.

As will be appreciated, in some embodiments, a user may be arranged to perform each of the process steps. For example, a user may measure the water and water surfactant into a container and thereafter move the vessel to the mixing station(s), and to other subsequent station (e.g., a high-energy homogenization station). In other embodiments, the system may be automated such that a robotic device is arranged to measure and add materials (e.g., fragrance materials and surfactants to containers), and to moves the container(s) between stations during the mixing, high-energy homogenization, and/or quality control steps.

In some embodiments, such as those described above, processes are provided for developing scented liquids such as those that may be vaporized by one or more systems described herein. For example, such scented liquids may be vaporized by one or more of the systems shown and discussed below (e.g., which show various systems, methods and elements used to vaporize scented liquids in one or more applications).

Historically, there have been many attempts at providing scents in various environments, such as theaters, computer environments, among other situations and locations. However, many of these technologies failed to reach widespread adoption. Also, some attempts have been made to extend scents technology to virtual reality environments, however, there is no common device available that is capable of rendering scents in such environments. Accordingly, the inventors have appreciated that there are no adequate commercially available devices capable of rendering scent information in an AR or VR environment.

According to some embodiments of the present disclosure, a system is provided that is capable of rendering scent information to a user. According to some embodiments, the inventors have recognized the benefits of having a device that could be used with existing Extended Reality (“XR”), Altered Reality (“AR”), or Virtual Reality (“VR”) headsets to render scent information to the user. Such scent information may be rendered by a game engine responsive to activities performed or experienced within the XR, AR, VR, or other types of environments. In other embodiments, such functionality may be incorporated within such headset devices.

Such a device, according to some embodiments, may be provided as a companion device or may be fully embedded in an Extended Reality (“XR”), VR or AR headset system (e.g., the well-known HTC Vive, Oculus Rift, Microsoft HoloLens, HTC's Gear VR among other devices and/or systems). The device, may, in some embodiments include a controller (or other type of processor) that is capable of communicating with a game (or content delivery) engine, operating system (e.g., Windows mixed reality, Google daydream) or other type of content delivery processor that produces AR and/or VR content.

In some embodiments, the device, sometimes referred to herein as an olfactory virtual reality or “OVR” device or system that provides olfactory stimuli, may include an aerosol generator or “AG” device for producing vaporized media to render scents. The AG device may include, for example, a piezoelectric vibration device that is used to produce scents corresponding to actions performed in an XR, VR or AR environment. That is, in some implementations, a user may interact with one or more game elements within a game program being executed by the game engine, and responsive to the interaction, the game engine may communicate a series of commands that cause a piezoelectric device of the OVR device to generate scents to be experienced by the user. In such embodiments, the generated scents may correspond to the actions being performed in the XR, VR, or AR environment.

According to some embodiments, the game engine is coupled to the OVR device via one or more communication channels such as a wireless interface (e.g., Bluetooth, WiFi, etc.). The game engine (or other type of content producer) may communicate with the OVR device using a stream of serial data, which when received by the OVR device, may be translated to scent commands that operate one or more piezoelectric elements of the OVR device.

In some embodiments, the OVR device further includes one or more detachable elements that contain a scent module. In some embodiments, the detachable element may include a vessel or other suitable container for containing the scent module. The detachable scent modules may, in some embodiments, include one or more scents that can be controlled by the game engine. In some embodiments, there may be a number of small scent modules, each associated with a separate piezoelectric element that can be addressed and used to render a scent to the user. The scent modules may be constructed using an element that contains one or more scents, which can be in the form of liquid, gel or solid scent media.

In some embodiments, the microcontroller or other processor type may control an amplitude of a piezoelectric device, which, in turn, may control airflow and scented media output that interacts with a corresponding detachable scent module. The volume of scent delivered to the user's olfactory organs may be controlled more accurately using such a control. Also, in some embodiments, a larger range of rendered scent strengths may be produced as a result.

In some embodiments, there may be one or more stages of piezo elements used to render scent information. As discussed further below, some elements may be used to provide fine control of the outputs of specific scents, while other elements may be used to perform primarily airflow movement, alone or in addition to fan elements or other air moving devices. In some embodiments, the piezo elements may or may not have separate vessels that contain the scent media. In some instances, the piezo elements may come preloaded with scent media. Some types of piezo elements may provide a replaceable form of scent media, such as a wick, insert or other media-containing element. In some embodiments, the piezo driven device vibrates liquid through a fine mesh to output an aerosol or other atomized output to the user's nose.

The piezo driven AG may take many forms, such as devices using vibrating mesh technology (“VMT”). For example, a ring-shaped piezo device formed around a plate with aperture holes having specified sizes may be used to vibrate a liquid into a fine mist that is dispersed in the air surrounding a user's nose. Such plates may be, in some embodiments, flat or formed (e.g., domed). In some embodiments and application types, the size of the holes may be less than 10 microns. As will be appreciated, the holes may have other suitable sizes. The holes may be formed of any suitable shape.

In another example, the piezo-type devices may include tubes of various shapes and sizes that have a piezo element surface attached to a tube surface, and which is arranged to vibrate and force the liquid into a mist through an aperture plate having holes. It should be appreciated that still other arrangements and types of piezo elements may be used in other embodiments.

In some embodiments, an arrangement of piezo elements (e.g., an array of piezo elements) may be used to provide scent information to the user. Such arrangements may be directly addressable via a controller or other device to control each of the piezo elements in the array. Some embodiments may use an array of piezo elements positioned near the user's nose to provide scent output directly to the user.

In some embodiments, a chamber may be formed near or around the user's nose to permit the user to receive the outputs of the piezo elements. The chamber may be formed, for example, using a housing that substantially surrounds the user's nose and that directs outputs of the piezo elements towards the user's nose. In some embodiments, the housing may be adapted to be mounted to an underside of an existing headset device.

According to some embodiments, the device includes a plurality of piezoelectric elements that are capable of being operated within a number of variable states. Such states may be controlled by a processor such as a microcontroller. The piezoelectric elements may operate as pumps that can be used to drive scents within channels that are positionable near the user's nose. In some embodiments, these channels may be configured in a variety of configurations using, for example, tubes or conduit, air reservoirs, vessels, and/or other physical constructs to obtain a system that disperses sent into or near the user's nose.

In some embodiments, the OVR device may include a processor and a serial input that receives an output provided by the game engine or other computing entity (e.g., other programs, systems, etc.). In some embodiments, an application programming interface (“API”) may be provided as a programmatic interface by which games and other external programs may be used to control and deliver scent information. By transmitting certain sequences of commands, the OVR device may be capable of delivering a scent output by controlling delivery of the variety of scented medium contained within the vessels. The variety of scented medium can be dispersed singularly or in combination to achieve a realistic sense of an object or environment. In some embodiments, the vessels can be designed to contain the different scented media in liquid, solid or even gel form. The vessels may also contain certain functionality or identifiers that allow them to be identified to the OVR system (e.g., what type of scent, level of media, etc.). In some embodiments, different combinations of vessels may be associated with different game formats. In some embodiments, each vessel may be designed to be changed out when the scented media is depleted.

The device, according to some embodiments, may be provided as a companion device or may be fully embedded in a Virtual Reality (VR) or Altered Reality (AR) headset system.

According to some embodiments, coupling devices are provided to attach the OVR device to various headset systems, such that outputs of the OVR device are positioned near the user's nose. In other embodiments, the OVR device features may be fully incorporated within the headset system. In one implementation of a fully integrated system, commands used to control OVR functions are integrated within the headset inputs provided by the game engine. In some embodiments, the OVR device also may be integrated with other inputs and outputs, such as blood pressure monitors, haptic feedback devices, heartrate monitors, eye movement monitors or other devices.

In some embodiments, an atomizer is provided for dispensing liquids into the air. In some implementations, a device may be provided for generating atomized fluid specifically, but not necessarily exclusively, for production of small droplets of scented oil and other fluid-based fragrances, among other types of liquids. In some embodiments, the device comprises a tube having a proximal opening and a distal opening, wherein media inside the tube is forced out of the proximal opening via an aperture plate.

In some embodiments, the tube further includes at least one piezoelectric plate that is attached to a face of the tube. The device further includes an aperture plate that is attached to the proximal end of the tube whereas the distal end of the tube is connected to a fluid supply source for supplying fluid through the tube to aperture plate at the proximal end of the tube. In some embodiments, the aperture plate may include a plurality of conical apertures that extend through the thickness of the plate. As will be appreciated, the apertures may have other suitable shapes in other embodiments.

In some embodiments, the device comprises a tube having a proximal opening and a distal opening. In such embodiments, fluid may enter the distal end and be forced out of the proximal opening via the aperture plate. In some embodiments, fluid may be existing within the tube and/or added via the distal end, such as by a mechanism to add fluid as the device operates and forces the fluid out. In some embodiments, the device is provided with the fluid located within the tube.

According to at least one aspect, a system may include a processor, at least one piezoelectric element controllably coupled to the processor, one or more scented media, and an interface adapted to receive one or more commands from an external content processor. In some embodiments, the processor is configured to, responsive to the received one or more commands, control the at least one piezoelectric element to deliver an output scent using the one or more scented media.

In some embodiments, the system may include one or more vessels that contain respective ones of the one or more scented media. In some embodiments, the one or more vessels each includes a corresponding piezoelectric element that are controllably coupled to the processor. In some embodiments, the one or more commands may include at least one command that selectively controls an identified piezoelectric element to render a specific scent. In some embodiments, the one or more commands may include a plurality of commands that selectively control more than one piezoelectric element to render a blended scent.

In some embodiments, the system may include a programmable interface through which the external content processor may control the at least one piezoelectric element. In some embodiments, the one or more commands may each specify a duration and intensity value associated with a respective scent. In some embodiments, the system further comprises a housing, the housing comprising a physical coupling to a headset capable of being worn by a user.

In some embodiments, the system may include hardware that delivers an olfactory output to the user. In such embodiments, the physical coupling may position the olfactory output of the system proximate to the user's nose. In some embodiments, the processor, the at least one piezoelectric element, the one or more scented media, and the interface may be part of a VR or AR device. In some embodiments, the one or more vessels that contain respective ones of the one or more scented media are detachable from the system.

In some embodiments, the commands from an external content processor are communicated responsive to an interaction of a user in an AR or VR realm. In some embodiments, the external content processor communicates proximity information to the system responsive to the user's interaction with one or more elements in the AR or VR realm.

In some embodiments, the at least one piezoelectric element may include a tube having a proximal opening and a distal opening, an aperture element coupled to the proximal opening of the tube, the aperture element having at least one aperture, a piezoelectric element attached to a surface of the tube, the piezoelectric element adapted to receive an electrical signal that causes the piezoelectric element to vibrate and induce a wave along a length of the tube that forces a medium through the at least one aperture. In some embodiments, the tube is at least one of a cross-sectional shape of a square, a triangle, an oval, a rectangle, a circle, other polygonal, or other suitable shape.

In some embodiments, the tube is adapted to receive the medium through the distal opening. In some embodiments, the medium includes at least one of a solid, a liquid and a gel. In some embodiments, the tube is adapted to receive a wick element that delivers a liquid medium to be dispersed. In some embodiments, the piezoelectric element forms a unimorph element with the tube.

According to some aspects, a computer-implemented method is provided including acts of receiving, via an interface of a scent generating device, a data element defining at least one scent to be rendered, processing, by a processor coupled to the interface, the received data element, controlling, responsive to processing the received data element, at least one piezoelectric element to deliver an output scent identified by the received data element. In some embodiments, the scent rendering device may include a plurality of scented media. In some embodiments, the received data element may uniquely identify the output scent among the plurality of scented media to be rendered.

In some embodiments, the data element may form a stream of data. In such embodiments, the method may include an act of processing a received stream of data, the stream of data defining a plurality of scents to be rendered. In some embodiments, the data element defining the at least one scent to be rendered may define a duration and an intensity value associated with the at least one scent to be rendered. In such embodiments, the method further may include controlling, responsive to processing the received data element, at least one piezoelectric element to deliver an output scent responsive to the defined duration and an intensity value associated with the at least one scent to be rendered. In some embodiments, the data element defining the at least one scent to be rendered may define a start command. In such embodiments, the method may include an act of processing, by the processor responsive to the start command, one or more scent rendering commands defined by the data element.

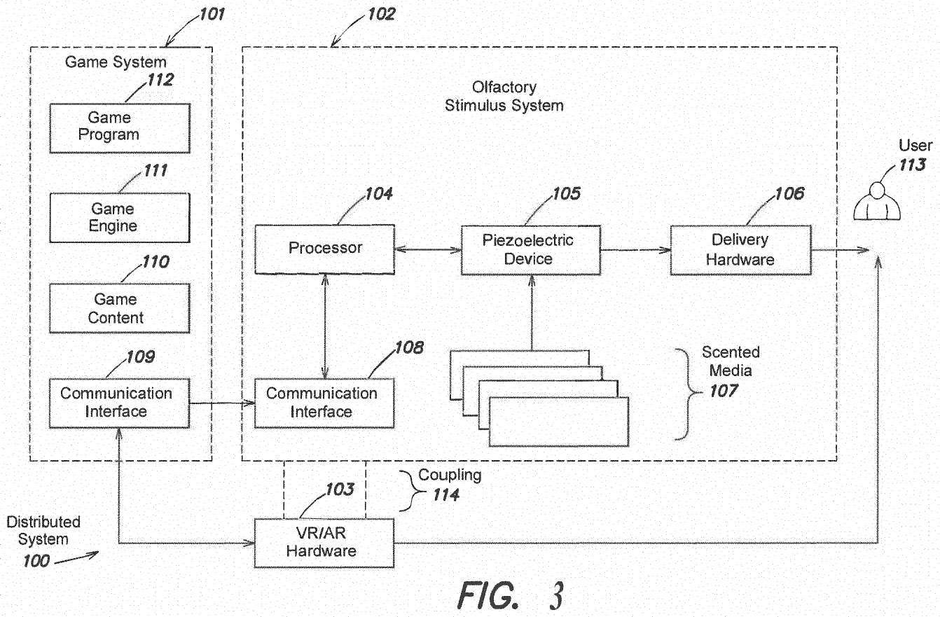

FIG. 3 shows a block diagram of a distributed computer system 100 capable of implementing various aspects of the present disclosure. As shown in this view, the distributed system 100 may include a game system 101, an olfactory stimulus system 102, and possibly separate VR/AR hardware, the combination of which are used to communicate information to a user 113.

In some embodiments, the game system 101 may include a game program 112, a game engine 111, game content 110, and a communication interface 109. In some embodiments, the game system 101 may use the game engine 111, which may include for example, any processors, code, and development platform used to write game programs (e.g., game program 112). According to various embodiments, game programs may be provided in an interface through which they can communicate with an olfactory stimulus system. Such interfaces may include, for instance, an API that defines commands and data structures for controlling the olfactory stimulus system 102. Further, the game system 101 may include one or more communication interfaces 109 which may be used to communicate to system 102. Such interfaces may include, for example, wired or wireless communication interfaces.

The olfactory stimulus system 102 may include a processor 104 that controls operation of system 102 functions. In some embodiments, the system 102 may include one or more piezoelectric devices (e.g., piezoelectric device 105) which may control the delivery of one or more types of scented media 107 for the purpose of rendering scent information to the user (e.g., user 113). The piezoelectric device 105 may deliver an olfactory output via delivery hardware 106. In some embodiments, delivery hardware may include, for example, vessels, interconnecting tubes, reservoirs, venturi elements, inlets, outlets, channels and/or any other active or passive delivery mechanisms.

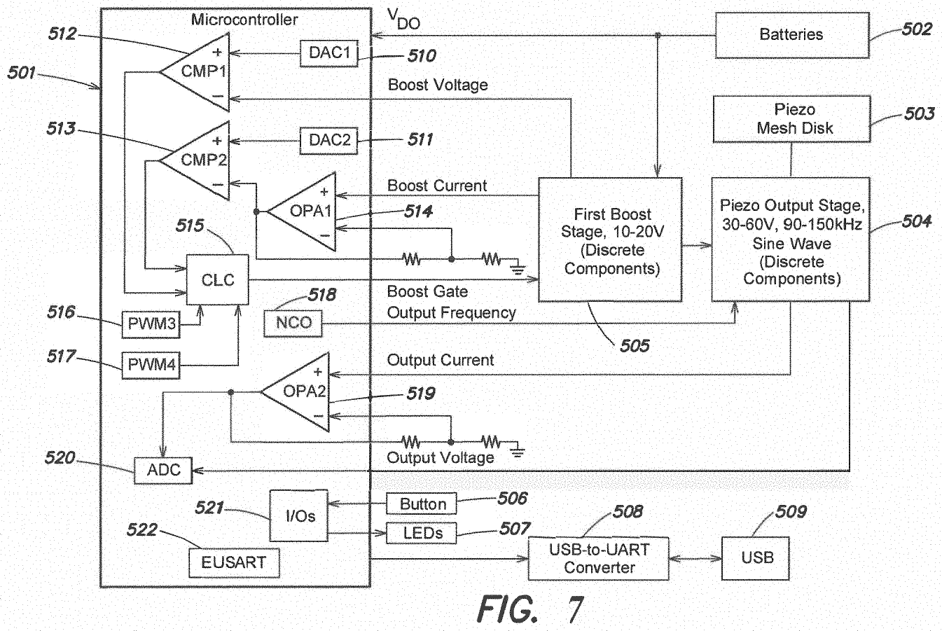

In some embodiments, the processor 104 may include a specially programmed microcontroller that performs certain specified control functions. One example of a specific control processor and circuitry is shown by way of example in FIG. 7. In some embodiments, the microcontroller (MCU) may include an ATmega328p Arduino-type controller. It should be appreciated, however, that other controller types may be used in other embodiments. Further, the microcontroller may also include some additional auxiliary components such as a frequency generator, digital potentiometer and one or more operational amplifiers which may be used to adjust voltage into a variable amplitude fixed frequency current that can be used to control a piezoelectric element.

In some embodiments, the olfactory stimulus system may be provided as part of an existing headset device, although, in other embodiments, the olfactory stimulus system may be provided as an additional device for existing VR/AR hardware (e.g., VR/AR hardware 103). In some embodiments, to accomplish this, a physical coupling 114 may be provided such that the olfactory stimulus system is positionable such that scent outputs may be provided to a user (e.g., user 113).

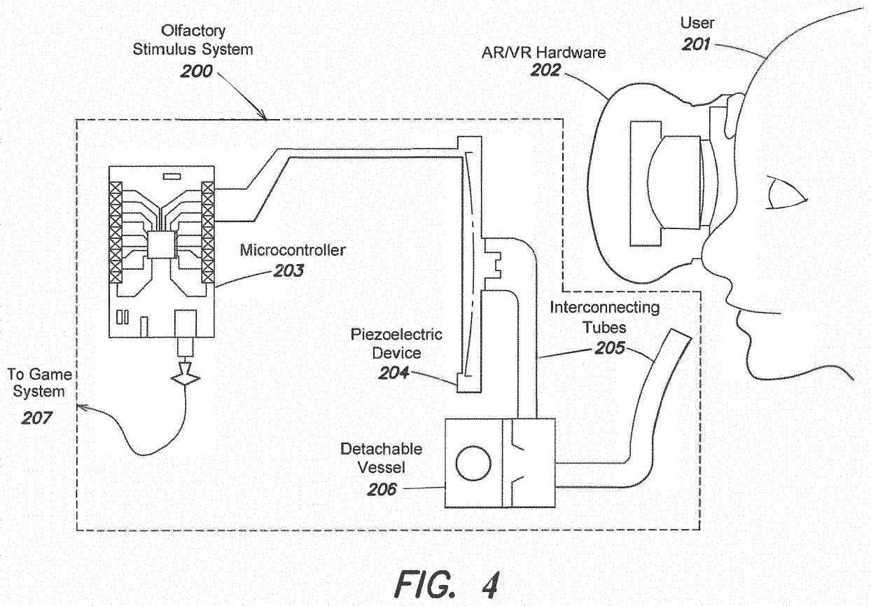

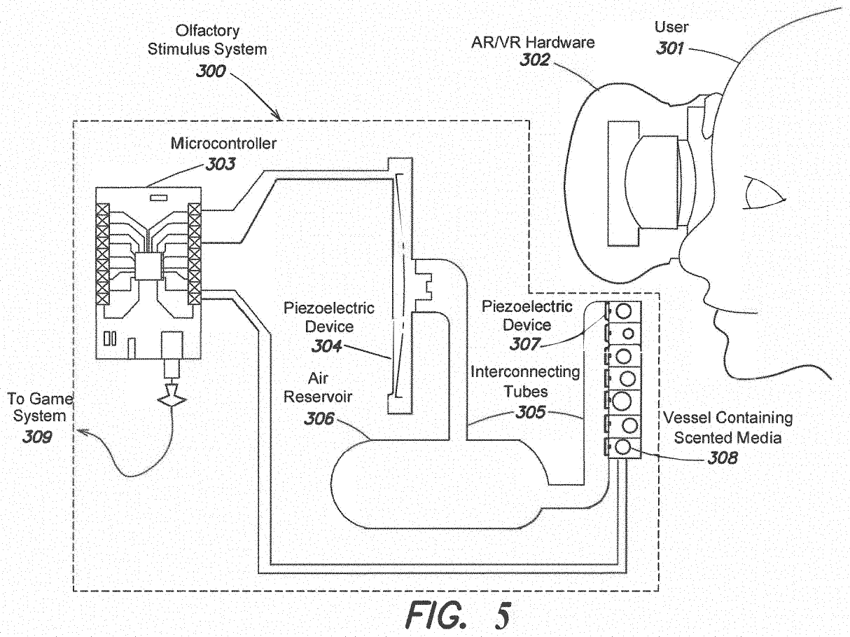

FIGS. 4 and 5 show various implementations of olfactory stimulus systems according to some embodiments. FIG. 4 shows embodiments in which the olfactory stimulus system 200 may be used with existing AR/VR hardware 202 to present scent information to user 201. As shown in FIG. 4, the olfactory stimulus system 200 may include a microcontroller 203 that controls a piezoelectric device 204. In some embodiments, the piezoelectric device 204 may act as a pump which blows air passed a detachable vessel 206 which contains scent media. As noted above, the vessel may include any suitable container for containing the scented media. In such embodiments, the air and/or scent particles may be routed in the system 200 via one or more channels, such as via one or more interconnecting tubes 205.

As will be appreciated, the tubes may have any suitable cross-sectional shape (e.g., round, square, triangular, polygonal, or other suitable shape). The tube may have one or more straight segments and/or one or more curved segments. In some embodiments, the one or more interconnecting tubes may include a single tube or may include more than one tube. In embodiments in which more than one tube is used, the tubes may be fixedly joined together.

According to some embodiments, piezoelectric components may be used to move air and, in some embodiments, diffuse liquids into a channel. Channels may be constructed using tubes manufactured using chemically resistant materials (e.g., brass or some other material). In some embodiments the channels may be manufactured using chemically resistant materials. In such embodiments, the chemically resistant material may counter the effects of water and possibly mild amounts of alcohol present within the scented media. According to some embodiments, such channel elements may be internally molded and/or printed elements.

In some embodiments, the detachable vessel 206 (and/or other elements of the olfactory stimulus system and embodiments described herein) may also be made from chemically resistant materials (e.g., glass, Plastic (PTFE, PEEK, UHMW, PTE, possibly HDPE chemically resistant variants), stainless steel, or other material(s) either alone or in combination with other materials).

As also shown in FIG. 4, the microcontroller 203 may be coupled to a game system 207 via one or more interfaces (e.g., a communication interface such as a wired or wireless connection (e.g., Bluetooth, Wi-Fi, or other type wireless communication protocol)).

FIG. 5 shows an alternative configuration of an olfactory stimulus system 300. Similar to the olfactory stimulus system 200 of FIG. 4, the system 300 of FIG. 5 may be used with existing AR/VR hardware 302 to present scent information to the user 301. As shown in FIG. 5, the olfactory stimulus system 300 may include a microcontroller 303, one or more piezoelectric devices (e.g. devices 304, 307) that may interface with a game system (e.g., game system 309), and one or more channels, such as reservoirs (e.g., air reservoir 306), tubes (e.g. interconnecting tubes 305), vessels (e.g. one or more vessels containing scented media 308). As illustrated in FIG. 5, in some embodiments, the olfactory stimulus system 300 may have a two-stage design where smaller piezoelectric elements are provided in addition to a main piezoelectric element that provides the majority of air movement.

In the alternative configuration shown in FIG. 5, separate piezoelectric devices may be provided for specific vessels that contain various scented media. For example, the microcontroller may be selectively controlled to activate certain piezoelectric devices to control delivery of particular scented media. As discussed further below, commands that specifically address particular piezoelectric devices may be provided such that the game system may control delivery of particular scents. For example, a first scent may be delivered during a first portion of a game and a second scent may be delivered during a second portion of the game.

In some embodiments, different vessels may contain different scents. In one implementation, vessels may contain active logic that communicate their information with microcontroller 303. For example, the active log may communicate what scents the vessels contain, a status, and/or a level of media. As will be appreciated, other information may be communicated via the active logic. In some implementations, collections of vessels or individual vessels may be removed and/or replaced when they are exhausted. As shown in FIG. 5, the system 300 may include an air reservoir 306 such that air pressure may be stored in a controlled manner and selectively delivered to individual vessels to provide a rendered output.

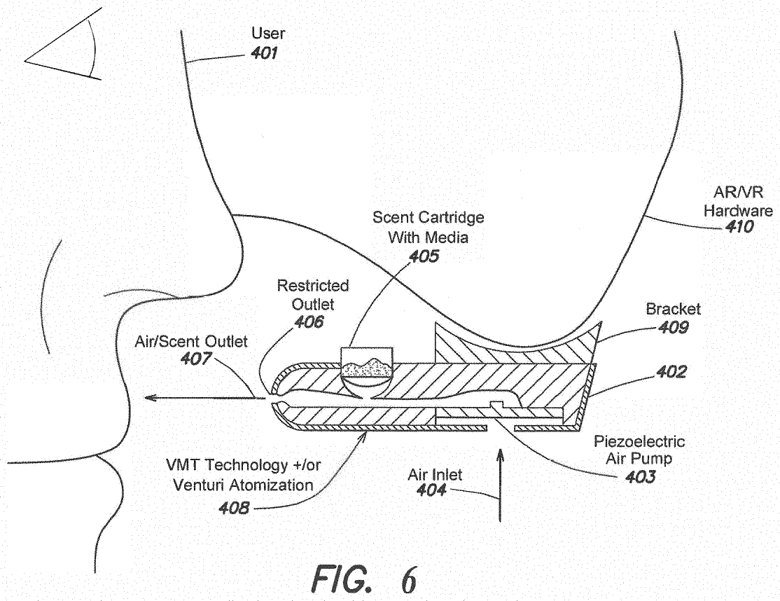

FIG. 6 shows another example device configuration that may be used alone or in connection with other embodiments. As shown in FIG. 6, olfactory stimulus device 402 may be connected to an existing AR/VR hardware 410 via a physical bracket 409. In some embodiments, the position of the device 402 may be adjustable such that that the olfactory output (e.g., air/scent outlet 407) may be positioned near a user's nose 401. In the configuration shown in FIG. 6, device 402 may include an air inlet 404, a restricted outlet 406, a piezoelectric air pump 403, and venturi technology. In some embodiments, the venturi technology may include an atomizer nozzle. In some embodiments, the piezoelectric air pump 403 may operate to pump air from the air inlet 404 within the chamber which mixes with an output of a scent cartridge having media (e.g., cartridge 405). In some embodiments, the mixture may be pumped through a restricted outlet 406 to the nose of the user.

FIG. 7 shows an example circuit in control function circuitry used to implement various aspects of the present disclosure. For example, a microcontroller 501 may be provided, which includes one or more digital to analog converters (e.g., elements 510, 511), one or more comparators (e.g. comparators 512, 513), and operational amplifiers (e.g. operational amplifiers 514, 519). In some embodiments, the circuit may be used to boost current and voltage and output gate frequency to operate a piezoelectric output stage (e.g., 504), which, in turn, may control a piezo mesh disk (e.g., element 503) which renders the scented output.

The microcontroller 501 may include one or more I/O ports to communicate information and receive information from various elements (e.g. button 506 and LEDs 507). In addition, the microcontroller 501 may include an element (e.g., EUSART 522) to communicate serial data to outside elements (e.g., such as by converting serially formed UART data to a USB output using a USB-to-UART converter 508 and USB interface 509). In some embodiments, the device may operate on its own power supply, which may include batteries 502 or another suitable power input.

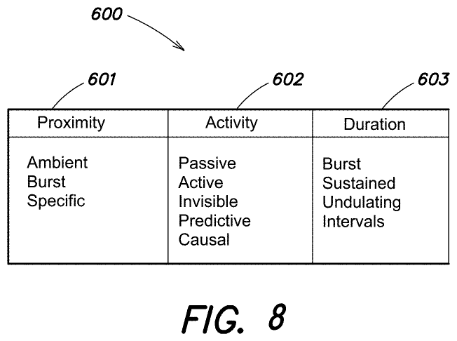

Embodiments disclosed herein may relate to ways of representing scent information in a distributed system and to encoding and decoding such information. FIG. 8 shows an exemplary implementation with illustrative scent classification information that may be used for communicating scent information in a distributed communication network. The inventors have appreciated that smell architecture may be important when it comes to creating a realistic experience, especially in an AR/VR environment such as those provided in virtual reality, altered reality or telecommunication devices using headsets or other devices.

According to various embodiments shown by way of example in table 600, various types of information may be used to classify or qualify scent information. In some embodiments, a particular scent may include proximity information 601, activity information 602, duration information 603, and appeal information.

In some embodiments, proximity information may be used to express how close the user or player is to an odorant object, such as within the AR/VR environment. In one embodiment, the proximity settings may dictate whether a smell is “on” or “off”. As shown in FIG. 8, proximity information may include Ambient information (e.g., the foundation), with the overall smell of a particular environment arranged to set an emotional tone. Proximity information also may include Burst information (e.g., walls, floors, lighting, furniture), with the smell of an object or collection of objects noticeable when passing within a particular distance, such as 1 meter. Finally, proximity information may include Specific information (e.g., appliances), with the smell of a specific object noticeable only when 12 inches or less from face

In some embodiments, activity information may be used to express the level of conscious interaction the player is having with a particular odorant object. It is appreciated that the level of conscious interaction may not necessarily be directly linked to the proximity of the player to the object. Instead, the level of conscious interaction may be linked to activity being performed. As shown in FIG. 8, activity information may include Passive, Active, Invisible, Predictive, and Casual.

In some embodiments, passive activity information may include bursts. For example, Passive activity information may include smells that are activated by passing by an object that may not necessarily be interactable but may play a role in creating ambience or foreshadowing in the narrative. Active activity information may include information from when the player interacts with an object deliberately. For example, the player may interact with objects for curiosity, to gain information, and/or to solve a puzzle. Invisible activity information may include a smell that may be released upon performing a specific action like opening a bottle or drawer. In some embodiments, invisible activity information may allow for circumventing the standard proximity protocols. Predictive activity information may include predictive smells that may come on the breeze or around a corner or from behind a closed door. Predictive activity information may include, for example, a predictive smell such as fire and/or smoke, or be something ever changing to promote a sense of doom. Casual activity information may include the effects when the user takes an exaggerated breath in.

As also shown in FIG. 8, duration information may be used to express how long the smell is being activated in the hardware and may include bursts, sustained, undulating, and Intervals. In some embodiments, Burst duration information may include a release of a predetermined time (e.g., 1 second) of a single or series of heavily diffusive aromas. In some embodiments, navigating through the VR environment may include navigating through different bursts. In such embodiments, pockets of scents may be experienced in succession through space and time may create an aromatic tapestry potentially as rich as the visual one.

Sustained duration information may include a slow continuous release of scent to either block outside odor or create subconscious reaction. In some embodiments, the sustained Duration information may be very faint. Undulating Duration information may include a single smell meant to be experienced over a longer period of time. In some embodiments, due to the “habituating effect” of the olfactory system, it may be necessary to increase and decrease intensity in a set predictable manner. Intervals duration information may include a way to mimic smell intensity by modulating rapid microbursts.

As will be appreciated, other types of encoding scent information may be used in other embodiments, and some embodiments may use different types of encoding.

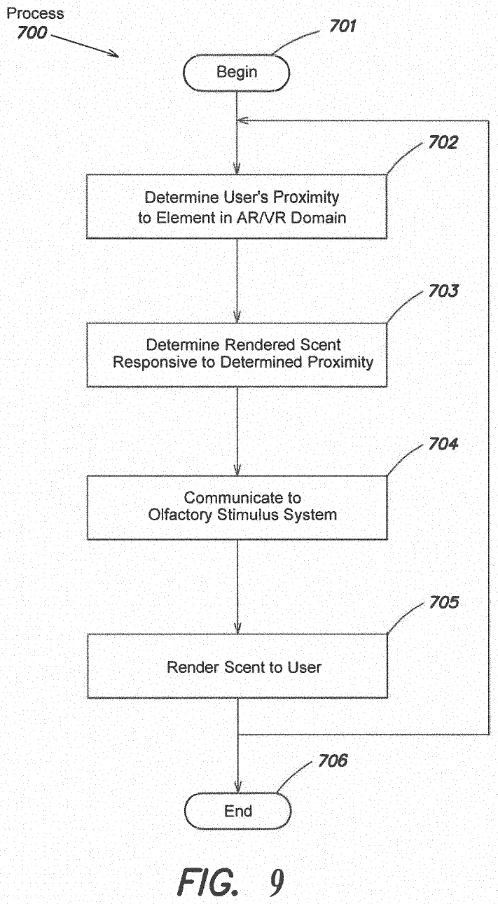

FIG. 9 shows an example process for rendering scent information according to various aspects of the present disclosure. As shown in this view, the process 700 begins at block 701. At block 702, the user's proximity in relation to an element in an AR/VR domain is determined. For example, the game engine, while executing the game code, may monitor the user's proximity to one or more virtual elements such as environmental elements, game elements, or another surface or object. At block 703, the system may determine a rendered scent responsive to the determined proximity between the user and the virtual element. For example, if the user is within a certain proximity of a surface that has a scent associated with it, the executing software may determine a scent to be “played” to the user at some point in time during the game execution or other contact rendering to the user. At block 704, the system communicates control information indicating the sent to be rendered to the olfactory stimulus system. Such information may include any type of encoding information, such as a duration of a scent to be rendered, an intensity value or other information. Such information may be transmitted over a wired or wireless communication link between a content providing system and the olfactory stimulus system. At block 705, the olfactory stimulus system may render the sent to the user. At block 706, the process 700 may end. As will be appreciated, and as shown by the upwardly directed arrow in FIG. 9, the process may work as a continuous loop as the user is experiencing the AR/VR content, and the process may return to block 702 instead of ending.

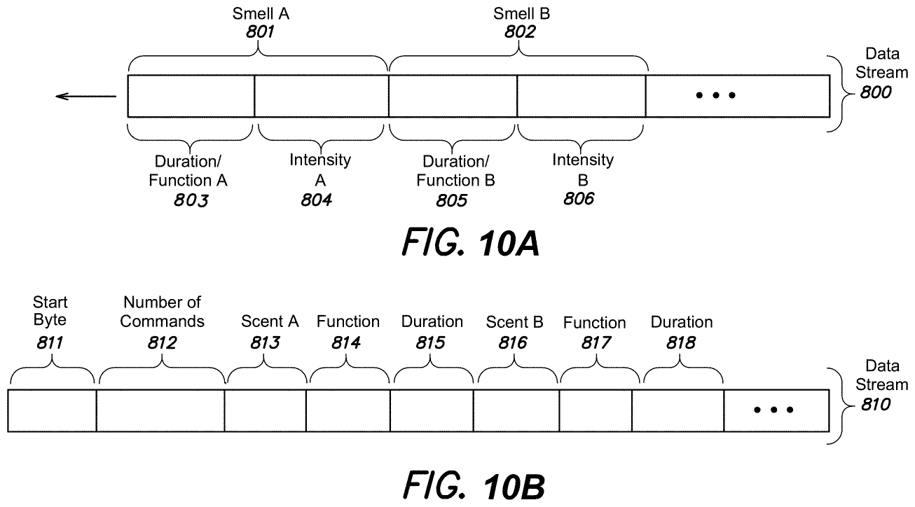

FIG. 10A shows an example format for communicating scent information according to various embodiments of the present disclosure. As shown in this figure, the olfactory stimulus system may be capable of receiving a data stream (e.g., data stream 800) sent from a game engine or another content providing system for the purpose of communicating smell information. As shown, the data stream may include one or more pieces of information that correspond to particular smells to be rendered to the user.

For instance, a portion of information corresponding to smell A (e.g., item 801) may be transmitted serially from the content provider to the olfactory stimulus system. Data element 801 may include a number of fields, characteristics, and/or values that qualify a particular smell. Data element 801 may include specific information that identifies which smell to be played, what duration, and in what intensity. Data element 801 also may include additional information encoded that reflects how the sent is to be delivered to the user. In some embodiments, data element 801 may include a duration and/or function for smell A 803. Such information may include a value that specifies the duration, as well as a specific identification of smell A. Further, data element 801 may include an intensity value for smell A 804 that may numerically represents a played intensity of the identified smell. As shown in FIG. 10A, the system may be capable of transmitting multiple smells (see smell B labeled 802). In such embodiments, the data stream also may include information regarding the duration and/or function of smell B 805 and the intensity information of smell B 806).

FIG. 10B shows another example format for communicating scent information according to various embodiments. In some embodiments, similar to the system described above with reference to FIG. 10A, the olfactory stimulus system of FIG. 10B may be capable of receiving a data stream (e.g., data stream 810) sent from a game engine or another content providing system for the purpose of communicating smell information. As shown in FIG. 10B, the data stream may include one or more pieces of information that correspond to particular smells to be rendered to the user. Notably, data stream 810 may be a different format which is communicated to the olfactory stimulus system when the scent is needed such that data is not continually sent and need not be processed when scent should not be present. In such a format, the data stream 810 (e.g., a partial stream or finite string of data) may be sent to the olfactory stimulus system.

As shown in FIG. 10B, Data stream 810 may include a start byte 811 that appears at the start of the message and which indicates to the olfactory stimulus system (e.g., a microcontroller operating the olfactory stimulus system) to start processing remaining bites and the string or partial stream of data thereafter. In some embodiments, in a resting state, a microcontroller of the olfactory stimulus system may be constantly for receipt of a start byte (or other header type or indication). The second portion of the message may include a number of commands 812 which indicates the number of scents in the stream, and which indicates how long the stream will be. Following data element 812 are the scent indications to be rendered (e.g., scent A, scent B, etc.). Each of the scent indications may include, for example, a scent label or designation (e.g., an encoded form of Scent A placed within data element 813), a function state of the scent (e.g., an intensity, delivery pattern, etc. for the scent encoded in data element 814), and a duration of the scent (e.g., element 815). Each of the various scents to be rendered also may include respective function and duration information encoded within the data stream.

It should be appreciated that smell information may be communicated in real time between entities for the purpose of delivering a realistic environment. Such information may be transmitted in parallel with AR/VR environment information, and in some embodiments, there may be a coordination protocol that synchronizes such information.

FIG. 11 shows an example software architecture according to various embodiments. In some embodiments, game program 902 and game engine 903 may be capable of communicating to the olfactory stimulus system 904 via an olfactory API 901. Olfactory API 901 may provide functions, interfaces, and parameters through which the game program 902 may communicate with the olfactory stimulus system 904. Further, in some embodiments, communication through the API may be bidirectional such that information may be sent to and also received from the OVR system. For example, a status of the OVR system may be communicated and may be visible to a content providing application. In an illustrative example, whether the OVR system is functioning, has appropriate and suitable levels of media, etc. may be provided to from the OVR system to another computing entity.

In a illustrative example, when someone encounters an object in VR there are things that occur on the game software/drivers side of the game and then there are things that happen on the hardware/firmware side of the game. On the software side, a player interacts with an object based on proximity to that object. The user's proximity to an object generates a value in the gaming engine. Other objects may distort that proximity value such as a wall or wind effects.

The value (whether or not it is modified) is then formatted into a string of characters by the API. That string of characters is then passed on to the microcontroller via USB or Bluetooth or LAN/WAN/Wi-Fi or any other digital wired or wireless communication link. In one example, the system may be connected via USB. As will be appreciated in view of the above, the string's length is determined by the multitude of scents. For example, the more scents there are to be rendered, the longer the data string sent over the digital connection.

On the hardware side, the string of characters may then relayed to the microcontroller and may be interpreted by the firmware (e.g., residing on the memory of the controller). The firmware selects a mode in which the smell may be delivered and then executes an amplitude on the piezoelectric value system(s) which is based on the proximity value generated from the software side. In one implementation, the entire process can be performed about 10-100 times per second and updates the amplitude of the scent as a user interacts with the VR environment and the predetermined or tagged objects in that environment. VR objects can be tagged during the development of the game by a game designer or post compilation of a game through the use of computer vision algorithms during game play.

It should be appreciated that the system, mechanical implementation, software and controls may have a number of features that are usable either alone or in combination with other features. For example, in some embodiments, the system may be capable of limiting “brown smell” or residual smells produced as a byproduct of playing previous smells. One example process for eliminating brown smell includes several methods. This first method may include using scent formulas and controlled atomization sizes which are highly dispersive and do not stick to surfaces very well. Without wishing to be bound by theory, this may ensure that the scent may clear away in a relatively short amount of time. A second process may include restricting the outlet size orifice near the scent cartridge which creates a passive high-pressure area. In some embodiments, this may function as a passive gate to keep additional scent molecules or atomized clumps from exiting the outlet when the piezoelectric devices are in a resting state. Essentially, this function may act as the brakes to the scent delivery mechanism. The third function may be to maintain control over the particle release size (nominally 20-2 um in size). Maintaining particle size may be accomplished, for example, through a VMT, venturi and/or other dispersion mechanisms. It should be appreciated that other features may be provided according to other implementations.

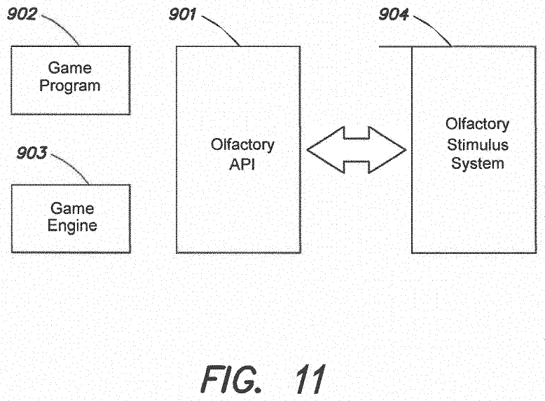

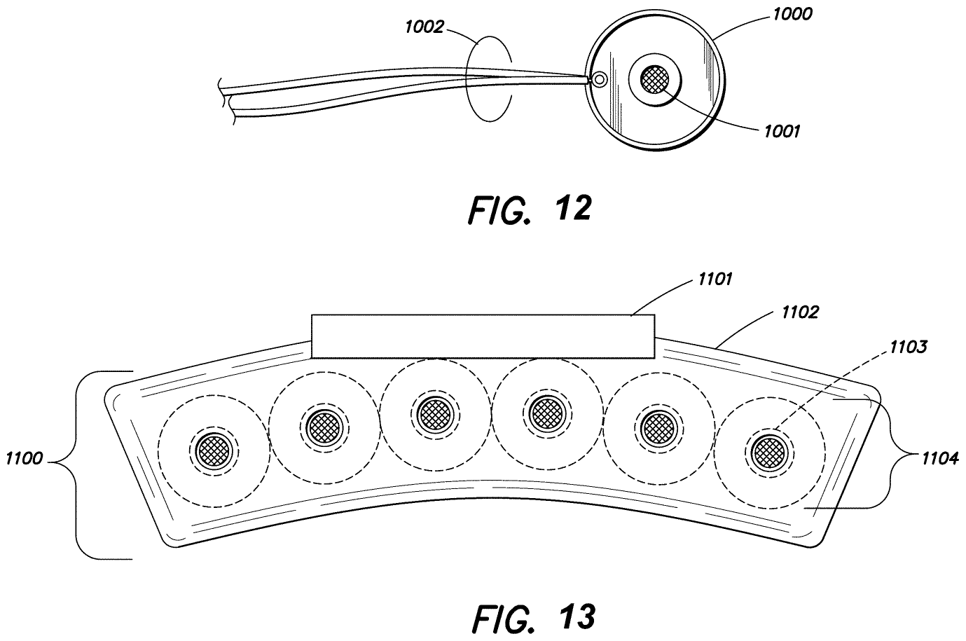

FIG. 12 shows another example device that may be used to render scent according to some embodiments. For example, FIG. 12 shows a piezoelectric device 1000 that may be used to render scent information. Device 1000 may be relatively small in size (e.g., 1-2 cm in diameter, or other size) such that it may be used in a personal scent rendering device, such as that shown by way of example in FIG. 13. Device 1000 may be circular in form, and include an area 1001 where scent is released. As will be appreciated, the device may have other suitable shapes (e.g., square, rectangular, triangular, etc.) in other embodiments. Device 1000 may include scent media either embedded within the device in some embodiments. The device 100 also may be capable of receiving scented material from a channel, or reservoir (e.g., in liquid form). Device 1000 may be operated by providing an activating signal through one or more electrical leads (e.g., leads 1002).

FIG. 13 shows an example device 1102 that may use one or more devices to render various scents according to some embodiments. In some embodiments, the device 1102 may be adapted to receive one or more piezoelectric elements such as those shown by way of example in FIG. 12. Further, device 1102 may be adapted to attach to an AR/VR headset (e.g., AR/VR hardware 202 in FIG. 4). For instance, device 1102 may be adapted to mount to an AR/VR headset via a mounting plate 1101. Device 1102 may be affixed to the headset via one or more attachment elements such as screws, mounts, adhesive elements, or similar elements. In some embodiments, the device 1102 may be fixedly attached to the AR/VR headset, although, in some embodiments, the device also may be removably attachable to the headset. In some embodiments, the device 1102 may include one or more openings 1103 through which scent is rendered. Because device may be mounted near a lower surface of the headset, the openings of device 1102 may be positioned near a user's nose. Device 1102 may be arc-shaped such that the openings are positioned substantially around an area near the user's nose. As will be appreciated, the device may have other suitable shapes in other embodiments.

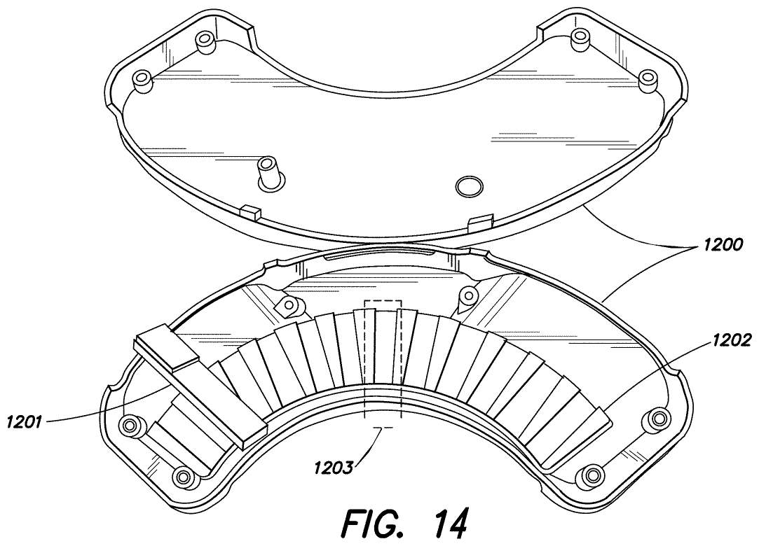

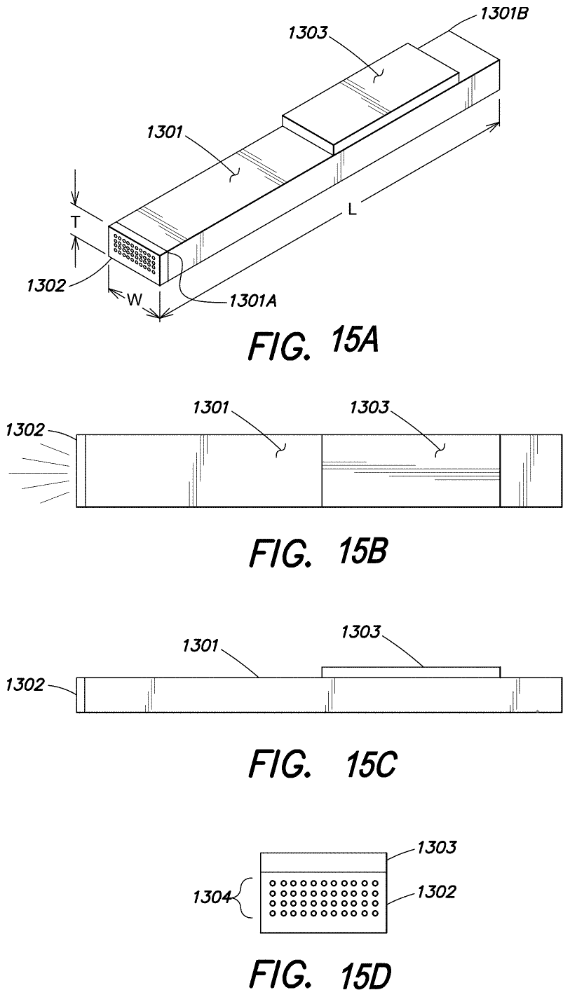

FIG. 14 shows another device 1200 that many be used to render various scents according to some embodiments. Similar to device 1102, device 1200 may be arc-shaped and may be adapted to be attached to an AR/VR headset. Also, device 1200 may be adapted to receive one or more piezoelectric elements (e.g., piezoelectric element 1201). In some embodiments, the piezoelectric elements may be rectangular in shape, such as those discussed with respect to FIGS. 15A-15D, and they may be configured to atomize a fluid and project the atomized fluid out of an end of the tube towards the user's nose. Several piezoelectric elements may be arranged in an arc of the device 1200. As will be appreciated, the device may have other shapes in other embodiments, with the piezoelectric elements being arranged in another suitable manner.

The piezoelectric elements may be held in channels (e.g., channel 1203) by a holding element 1202 (e.g., a holder). In some embodiments, the holding element may be manufactured using a rubber-like material to isolate the piezoelectric elements and their vibratory effects from one another and the main housing of device 1200. In some embodiments, the piezoelectric elements are sandwiched between several holding elements, thereby positioning and holding the piezoelectric elements within their respective channels. As will be appreciated in view of the above, the piezoelectric elements may be adapted to render different scents. Each of the elements may be selectively activated by a controller that sends activating signals to a particular selected element.

FIGS. 15A-15D show embodiments of a device for generating atomized fluid according to the present disclosure. As shown in these views, the device may include a rectangular tube 1301 having a cross-sectional shape, a width W, a depth T and a length L. In some embodiments, a piezoelectric plate 1303 may be attached across the width W of the tube. In some embodiments, and as shown in FIG. 15B, the piezo electric plate 1303 may extend along a portion of the length of the tube. For example, a length of the plate may be less than about half of the length of the tube. As will be appreciated, in other embodiments, the length of the plate may be the same as the length of the tube, with the plate extending along the entire length of the tube.

In some embodiments, the piezoelectric plate 1303 may be attached to the rectangular tube 1301 via glue, epoxy, solder or other adhesive. Other suitable adhesives, and other suitable attachment method may be used in other embodiments. It should be appreciated that although a rectangular tube is shown, other shapes of tubes may be used (e.g., circular, triangular, square, etc.). It should be further appreciated that although a rectangular plate is shown, other shapes may be used. Further, although the tube and plate are shown as having the shape, the shape of the tube and the shape of the plate may differ. Also, although a single plate is shown as being attached to the tube, in other embodiments, more than one piezoelectric plate may be attachable to the tube.

An apertured plate 1302 may be attached to an end of the tube 1301A while a second end of the tube 1301B is open and is configured to receiving a fluid and supplying the fluid to the aperture plate 1302 through the tube. In such embodiments, the piezoelectric plate 1303 may be connected to a circuit that generates an electrical signal at a frequency that is equal to the resonance frequency of the tube and in an amplitude that is sufficient to produce a flow of atomized droplets.

The electrical signal may be, in some embodiments, an alternating signal that is applied to contacts of the piezoelectric plate 1303.

In one embodiment, the tube is made of brass and has a width of 6.35 mm, a depth of 3.125 mm, and a length of 40 mm, with a resonance frequency of 50,000 Hz. It should be appreciated however, that other dimensions, configurations and resonant frequencies may be used. For example, in other embodiments, the tube may be between about 0.005 mm and about 14 mm (e.g., between about 5 mm and 7 mm in width), between about 0.002 mm and about 8 mm (e.g., about 2 mm and 4 mm) in depth, and between 40 microns and about 80 mm (e.g., about 13 mm or between about 38 mm and 42 mm) length.

In some embodiments, the piezo element and tube form a unimorph device including an active layer (e.g., the piezo element) and an inactive layer (e.g., the tube surface). One implementation may include a tube having a rectangular or square in shape. In some embodiments, a pinching and/or squeezing mechanism may be used to deliver liquid via the piezo element. In other embodiments, such as those disclosed herein, a medium (e.g., a liquid) may be aerosolized via a perpendicular acoustical waves induced by the piezo element.

As will be described, there are several ways in which the medium (e.g., liquid) may come into contact with the piezoelectric plate for aerosolizing the liquid. In some embodiments, the medium may be arranged to be free in a housing. For example, the medium (e.g., liquid) may be free in the tube and capped at the end opposite the aperture plate end to seal the medium inside. In such an example, the vibration pattern forces the liquid in contact with the apertured plate, with the aerosolized particles emitted therefrom.

In other embodiments, the medium may come into contact with the apertured plate via a wick. For example, the medium may be placed in the tube and capped in with the medium (e.g., liquid) to force the correct capillary action to move the liquid to aperture plate in conjunction with the vibration. In some embodiments, the wick may be shaped to fill the area within the tube (e.g., a rectangular, tubular, or square shape). In some implementations, the wick element may be a replaceable item, and may be accessible to be replaced. The wick may also be part of or coupled to a reservoir that holds the medium (e.g., liquid) to be dispersed. The wick may be, in some embodiments, bidirectional or unidirectional wicking material made out of, for example, natural fibers and/or synthetic fibers including cotton, polyethylene, nylon, metal, graphene, among others.

In still another embodiment, the medium may come into contact with the apertured plate via a cartridge. In such embodiments, the cartridge may be of a custom design is be inserted into the back to the tube with a connection point to the tube and plate. The cartridge may, or may not, use a wick or material that has a wicking property in some embodiments.

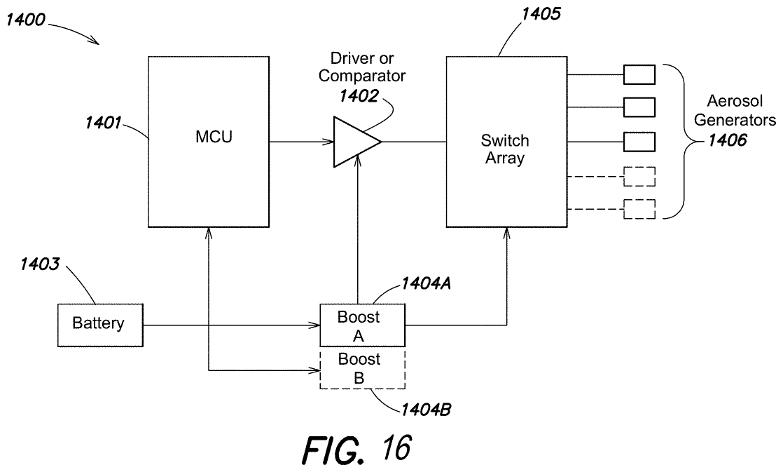

FIG. 16 shows an alternative control system according to some embodiments. In particular, one or more alternative control systems may be used in some embodiments where the piezo device includes one or more tube structures arranged in an array. The circuit may operate, for example, similarly to the system described above with respect to FIG. 7, which performs similar functions. In particular, a device driver circuit may be used to selectively activate different piezo elements (e.g., in an array) according to what scent is addressed (e.g., within a received stream of commands).

In some embodiments, generally within the driver circuit shown in FIG. 16, a microcontroller generates a frequency which may be then amplified in power greatly in order to drive selected piezo elements. Switches may be used to control the activation of the amplified power signal. The signal itself may be, for example, a signal of a fixed frequency with a 50% duty cycle. However, it should be appreciated that parameters of the signal (e.g., shape, length, height, pattern, etc. of the signal waveform) may be selectively varied to produce different intensities and lengths (e.g., duration) of scent production. Further, it should be appreciated that a DC signal may be used which includes positive signals or alternatively an AC signal may be used consisting of both positive and negative signals.

FIG. 16 shows a general circuit design which may include several subcomponents such as a battery 1403, a microcontroller (e.g., MCU 1401), a power conversion “boost” (e.g., via boost device A, boost B, labeled 1404A, 1404B) and a switching array 1405. Optionally, a driver or comparator (e.g., a MOSFET comparator, e.g., element 1402) may be used to drive the logic coming from the MCU to a higher or lower power level to drive the switching array. Also, optionally, a secondary power conversion may be used in order to provide a power source used to drive a second logic level voltage. The switching array 1405 is adapted to receive serial signal and convert that signal into the actuation of a specific channel. Each channel coming from the switch array is used to drive each of the individual aerosol generators (e.g., generators 1406). In some embodiments, the array should be sufficiently fast and rated for the appropriate voltage and current in order to be able to drive the aerosol generators in a real-time manner.

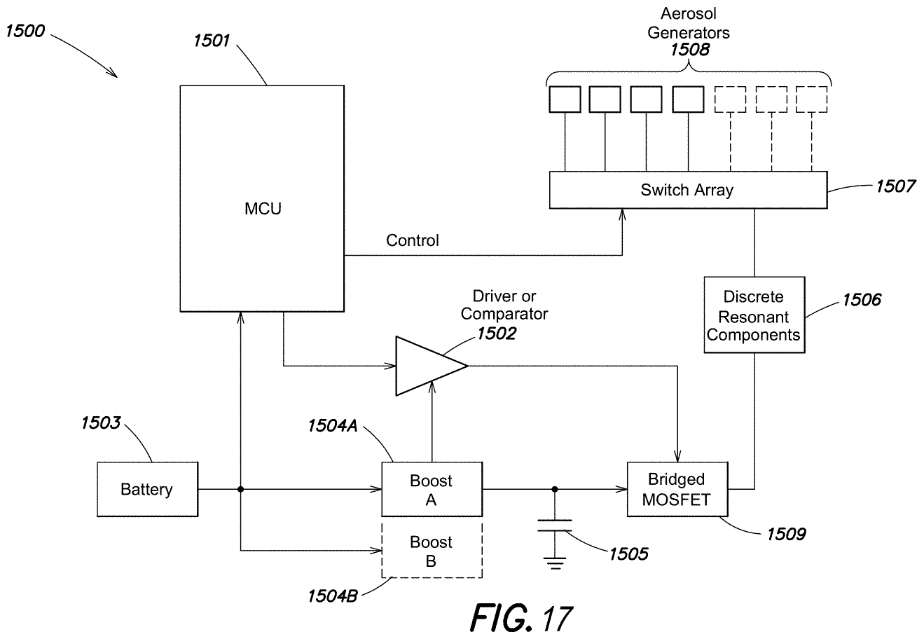

FIG. 17 shows another alternative control system according to some embodiments. In some embodiments, as shown in FIG. 17, the general circuit design may include includes several subcomponents such as a battery 1503, a microcontroller (e.g., MCU 1501), a power conversion “boost” (e.g., via boost device A, boost B, labeled 1504A, 1504B), a bridged MOSFET (e.g., element 1506) and a switching array (e.g., switching array 1507). Optionally a driver or comparator (e.g., a MOSFET comparator, labeled 1402) may be used to drive the logic coming from the MCU to a higher or lower power level to drive the switching array and or the bridged MOSFET. In some embodiments, optional discrete resonant components (e.g., discrete resonant components 1506) such as capacitors/inductors may be used for further power amplification and signal smoothing. In the circuit shown in FIG. 17, the bridged MOSFET takes signals from the microcontroller and then amplifies that signal to a higher power level. In such embodiments, the signals are typically in the form of a timed frequency with a duty cycle. In some embodiments, the switching array may then open a channel in which the power signal coming from the half bridge can then actuate the aerosol generators with the assistance/amplification of the resonant components.

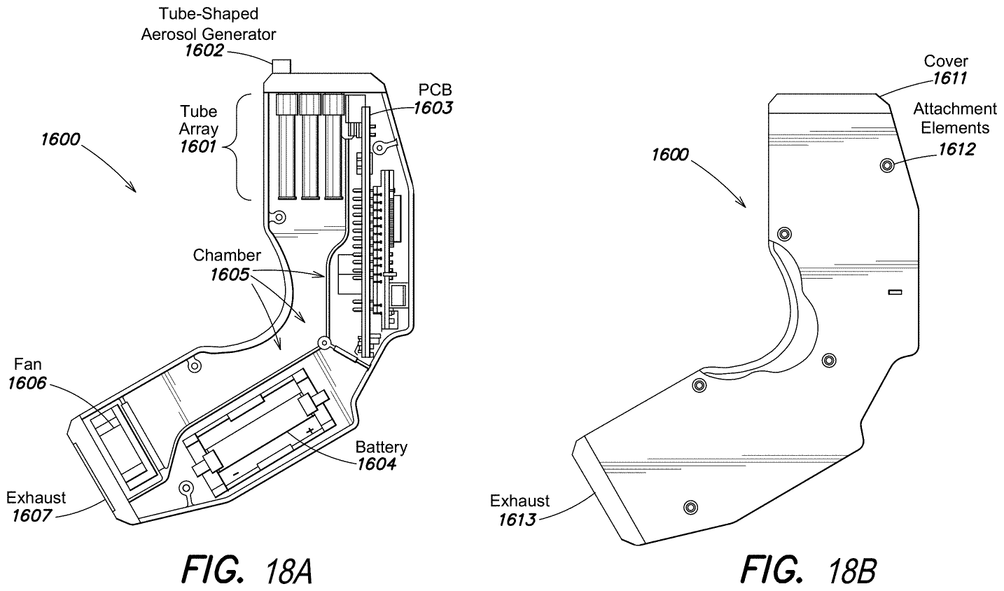

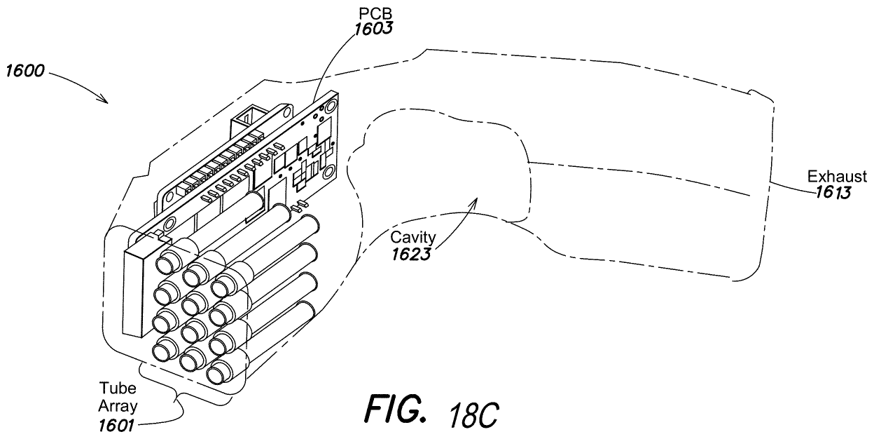

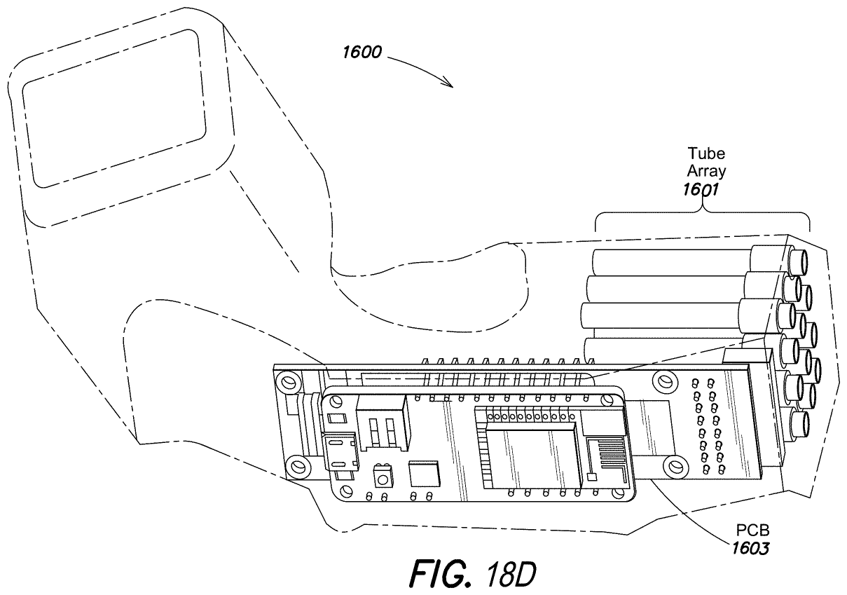

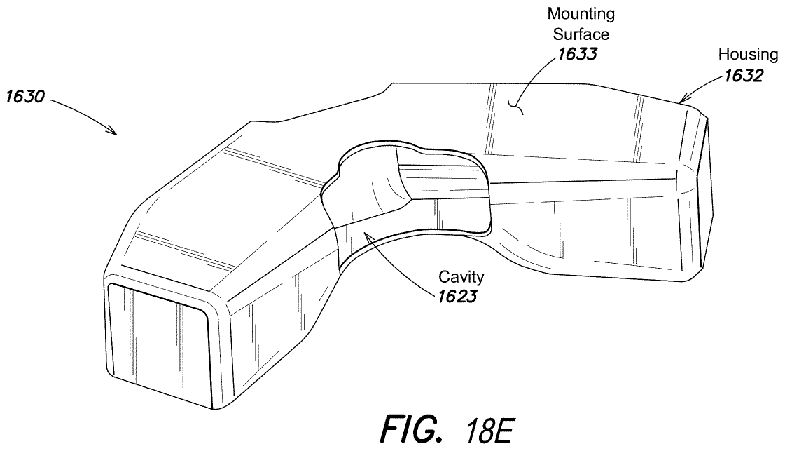

FIGS. 18A-18E show various views of an example olfactory stimulus system according to some embodiments. As shown in FIG. 16A, the olfactory stimulus system 1600 may include a device with an L-shaped housing with a number of components similar to those discussed above with reference to FIG. 3. For example, the olfactory system 1600 may include one or more piezo elements and in some embodiments, the Piezo elements take the form of tube-shaped aerosol generators 1602.

In some embodiments, the piezo elements are arranged within a tube array 1601. The piezo elements may be electrically connected to a PCB 1603, which may include one or more circuit elements such as those discussed above with reference to FIGS. 16-17. In some embodiments, system 1600 may include a battery 1604 that is used to power one or more of the components and generate signals that may be used to drive the production of scent by one or more aerosol generators. Outputs of the tube array 1601 may be positioned abutting a chamber 1605. in some embodiments, a user's nose may be positioned within an opening of the chamber to receive one or more outputs of the tube array (e.g., one or more scents). In some embodiments, the individual tubes, their media, and/or the tube array may be a removable and replaceable item. For example, in some embodiments, the tubes, their median and/or the tubular array may be replaced to renew exhausted media.

As also shown in FIGS. 18A, at an opposite end of the system, there may be an exhaust 1607, which may be used to remove sent from the chamber 1605. Near the output of the exhaust may be positioned a fan element 1606, or another suitable device for air moving device, which may be configured to move air in and out of the chamber from the exterior of the system 1600. In some embodiments, it may be useful to clear sent away from the chamber as well as mix outside air with scents produced by one or more of the aerosol generators.

As shown in FIG. 18B, the device 1600 may include a cover 1611 to enclose the elements within the device 1600. Cover 1611 is attachable to the remainder of the housing via one or more attachment element 1612. For example, the cover may be attached via screws or other fasteners. The cover also may be attachable via other suitable arrangements (e.g., an adhesive). In some embodiments, cover 1611 encloses the chamber whereby outside air is input via exhaust 1613 or sent is removed from the chamber via the exhaust 1613.

FIG. 18C shows a three-dimensional view of olfactory system of FIGS. 18A-18B. As shown in FIG. 18C, the system 1600 may include a three-dimensional tube array 1601 including, as shown, 12 different aerosol generators positioned within the array. In some embodiments, the tubes may be vibrationally isolated from each other such that vibration induced in one tube will not be translated significantly to another tube within the array. A housing of the device may include several openings, such as a cavity 1623 in which a user's nose is placed. As shown, a PCB 1603 and the tube array 1601 may be positioned opposite the exhaust 1613 located at the other side of the device.

FIG. 18D shows relative positioning of the PCB and tube array with respect to the housing and openings. FIG. 18E shows another view of the device, whereby only the external housing and viewable elements are seen. As can be seen in this figure, the housing 1632 forms the cavity 1623 in which a user's nose may be positioned. The device also may include a mounting surface 1633 which may be attached by one or more attachment mechanisms to an AR/VR headset, such that the device is positioned near the user's nose. For example, the mounting surface may be attached to the AR/VR headset via one or more fasteners, such as clips or bolts or screw or hooks, or another suitable fastener. It should be appreciated that elements shown in FIGS. 16A-18E (e.g., PCB elements, tube arrays, etc.) may be similar or the same items as those shown in the other figures. The elements shown in FIGS. 18A-18E also may be substituted with other elements as described herein.

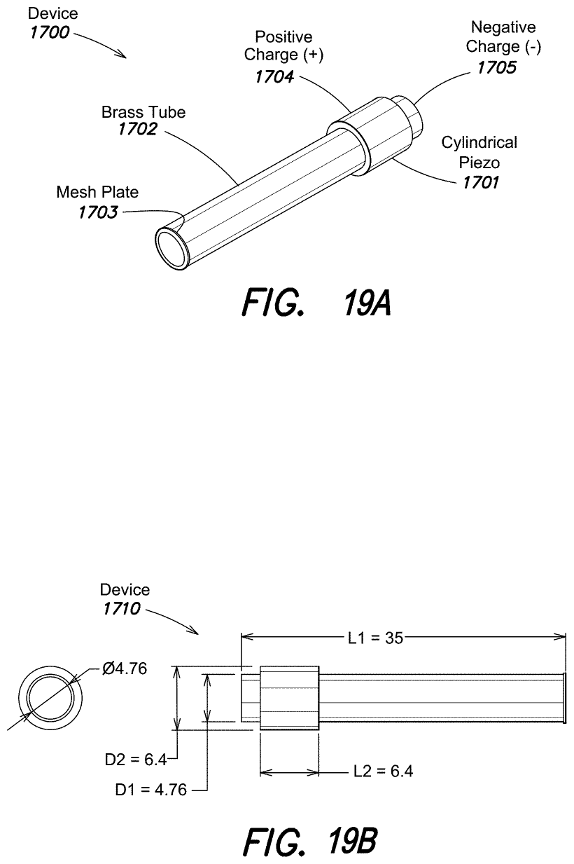

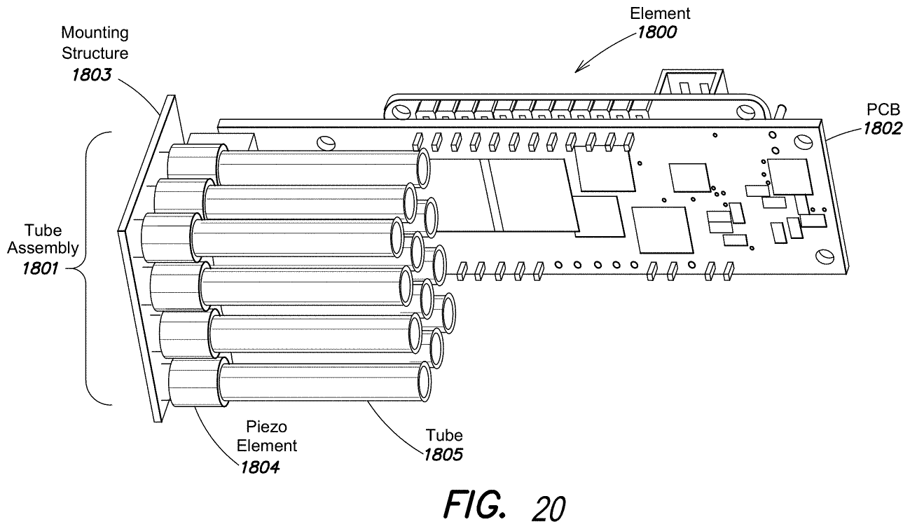

FIGS. 19A-19B show a device for generating atomized fluid according to some embodiments. As shown in FIG. 19A, in some embodiments, the device includes a round tubular device 1700. Although the deice is should as having a circular cross-sectional shape in FIG. 19A, the tubular device may have other suitable cross-sectional shapes (e.g., square and triangular) in other embodiments. As will be appreciated, the shape of the surrounding sleeve may be changed to correspond to the shape of the tube.

In some embodiments, the tubular device may be similar in function to the device discussed above with respect to FIGS. 15A-15D. In some embodiments, the device 1700 may include a tube 1702 having a length L1 and diameter D1. A piezoelectric sleeve 1701 may be attached at or near an end of the cylindrical tube, the piezoelectric sleeve having a respective length L2 and diameter D2. In some embodiments, the piezoelectric sleeve may be attached to the cylindrical tube via glue, epoxy, solder or another suitable adhesive.

Similar to the rectangular embodiment, an aperture plate (e.g., mesh plate 1703) may be attached to a first end of the tube while a second end may remain open and be configured to receive a fluid and supply the fluid to the aperture plate through the tube.

In some embodiments, the piezoelectric element may be connected to a circuit that generates an electrical signal at a frequency that is equal to the resonance frequency of tube and in an amplitude that is sufficient to produce a flow of atomized droplets. The electrical signal may be, in some embodiments, an alternating signal that is applied to contacts of the piezoelectric element (e.g., via positive charge 1704 being applied to the piezo layer and a negative charge 1705 being applied to the tube).

In one embodiment, the tube is made of brass and has a diameter of about 4.76 mm, and a length of about 35 mm, with a resonant frequency in a range of substantially 100-300 KHz. The piezo element may have a diameter of 6.4 mm and length of 6.4 mm. It should be appreciated however, that other dimensions, configurations and resonant frequencies may be used. For example, the range of the frequency that a particular device may function can vary from a relatively low frequency (e.g., 20 kHz) to a relatively high value (e.g., 1 GHz). Using the example circular tube devices described above, the resonant frequency may be determined to be in a range of 100-300 KHz. As will be appreciated, in embodiments in which the size (e.g., length and/or diameter) of the tube is decreased, the frequency increases. It should be appreciated that the resonant frequency depends on a number of factors and can be determined heuristically from testing the device.

In some embodiments, the piezo element and tube may form a unimorph device including an active layer (e.g., the piezo element) and an inactive layer (e.g., the tube surface). In some conventional piezo elements, a pinching and/or squeezing mechanism may be used to deliver liquids. In some embodiments as disclosed herein, a medium (e.g., a liquid) may be aerosolized via perpendicular acoustical waves induced by a piezo element. It should be appreciated that although certain shaped devices having certain dimensions are shown, other shaped elements having different dimensions may be used.

FIG. 20 shows a detailed view of a portion of an olfactory stimulus system (labeled element 1800) according to some embodiments. As shown in this view, the element 1800 may include a PCB 1802 having power and control circuitry that is used to selectively activate one or more piezo-based tubes within a tube assembly 1801. Each of the tubes (e.g., tube 1805) may be mounted on a mounting structure 1803. In some embodiments, the tubes are mounted to isolate them vibrationally from other tube elements. In some cases, spacers or other elements may isolate one or more tubes in the array. In some embodiments, piezo elements of each tube (e.g. piezo element 1804) may be positionally separated by adjacent tubes yet be mounted by a common electrical connection (e.g., via a separate PCB). In some cases, there may be isolation elements that isolate each tube from the mounting structure.

Although devices have been shown and described for devices may be used in gaming and entertainment applications, it should also be appreciated that the disclosed olfactory stimulus system may be useful in a number of different applications outside the gaming/entertainment area. For example, the system may be used for cognitive behavioral therapy. In some embodiments, cognitive behavioral therapists may a number of techniques to help their patients work through traumatic experiences, including exposure therapy and virtual reality. It is appreciated that conditions such as PTSD from war and sexual trauma are the hardest to overcome for one reason: smell. Such experiences are hardwired into our brains. In some embodiments, by integrating unique, curated aromas into therapy with VR, thousands of people may be helped to live normal lives and have normal relationships.

Devices also may be used for remote surgeries. For example, it is appreciated that people's sense of smell works more quickly and efficiently than all of our other senses combined. VR has the unique ability to allow surgeons to perform complicated surgeries remotely but still only effectively offers 2D sense of objects during complex procedures. Applicant has appreciated that by augmenting the surgeon's sense of critical areas with scent, the chance of error may be decreased without the need for the surgeon to break visual plane)

Devices also may be used for individuals with visual impairment. For example, for the visually impaired to participate in a VR or AR experience, various systems must take advantage senses other than eyesight.

Devices also may be used for forensics. For example, witnesses identifying the perpetrator is dangerously inaccurate and subject to implicit bias. Because of the direct link between scent, memory and emotion, VR may be coupled with scent creating a stronger, impartial, more just method of suspect identification, crime scene analysis and jury trials.

Devices also may have other therapeutic uses. For example, office, team, family, and relationship productivity may go up dramatically when people feel calm, rested and refreshed. For example, spending 10 minutes in scent enhanced, augmented reality can offer the same benefits as meditation, sleep or an hour of mindfulness.

Devices also may be used for sports medicine. For example, training in VR kick starts psychosomatic response (i.e., nothing can create a “Pavlovian response” more quickly and powerfully than scent training). In some embodiments, when an athlete is training for an event in VR, like the Tour de France, for example, aromatic stimuli may be created that increase or decrease heart rate, testosterone, or even a pain/pleasure response that may be recreated during actual competition.

Device also may be used for piloting. For example, as aeronautics and combat become more technologically advanced, any opportunity to make controls and feedback more intuitive to the pilot is paramount. It is appreciated that the very second the pilot has to pay attention to a gauge or otherwise take his eye off more important visual cues can have catastrophic events. Furthermore, in high stress combat situations quick decision making without hesitation is key. Because smell stimulates the limbic (e.g., fight or flight) portion of the brain before being processed by the pre-frontal cortex, it is appreciated that VR training simulations utilizing olfactory cues may increase response time, preserve focus and decrease stress responses in real life situations.

Devices also may be used for transposing senses and environmental conditions. For example, information of the environment such as temperature, humidity, radiation, and/or unscented poisonous gas. In some embodiments, exploration in environments that are dangerous or toxic to humans rely too heavily on sight and crude robotics. In such embodiments, by utilizing a VR/AR interface with a detection capability of scent that can be translated and communicated to an OVR system, the capability may be provided to explore the deep sea, radioactive sites, caves, and the like. In particular, human operators may receive and interpret data in real time in a much more meaningful way than ever before.

Devices may further be used for space applications. For example, astronauts often need to be able to sense physical phenomena on the edge of perception, e.g., gamma rays, x rays, oxygen and carbon dioxide levels, and an OVR system may be used to accomplish experiencing these environments.

In some embodiments, an atomizer is provided for dispensing liquids into the air. In some implementations, a device is provided for generating atomized fluid specifically, but not exclusively, for production of small droplets of scented oil and other fluid-based fragrances, among other types of liquids. In some embodiments, the device may include a tube having a proximal opening and a distal opening, wherein media inside the tube is forced out of the proximal opening via an aperture plate, examples of which are shown in FIGS. 15A-15D and 19A-19B. Such tubes may have any shape and size, such as rectangular or cylindrical tubes.

In some embodiments, the tube further includes at least one piezoelectric plate that is attached to a face of the tube. For example, the plate may be attached to an outer surface of the tube. The device also may include an aperture plate that is attached to the proximal end of the tube, with the distal end of the tube being connectable to a fluid supply source for supplying fluid through the tube to the aperture plate. In some embodiments, the aperture plate may include a plurality of conical apertures that extend through the thickness of the plate. As will be appreciated, the apertures may have other suitable shapes in other embodiments.

In some embodiments, the device may include a tube having a proximal opening and a distal opening, wherein fluid enters the distal opening and is forced out of the proximal opening via an aperture plate. In some embodiments, fluid may be disposed within the tube and/or added via the distal end, such as by a mechanism to add fluid as the device operates and forces the fluid out. In some embodiments, the device may be provided with the fluid disposed within the tube.

The device also may include a signal generator circuit capable of producing an electrical signal at a selected frequency and voltage. In some embodiments, when the frequency generator is connected to the piezo plate, cyclical stress waves may be generated by the piezo plate, which subsequently propagates along the length of the tube and produces oscillation which vibrates the aperture plate. This, in turn, may generate a flow of atomized liquid through the apertures. In some embodiments, at least one surface of the tube may have sufficient surface area to enable attachment of the piezo plate. In some embodiments, the tube may be rectangular in shape, and a surface of the piezo plate may be affixed to a substantial portion of a surface of the tube. In some embodiments, the piezo element is at or near a distal end of the tube, which may allowing the stress waves to travel more significantly towards and, in some embodiments to, the proximal opening of the tube. As will be appreciated, the piezo plate may be affixed at other locations along the tube, such as near a central region of the tube.

In some embodiments, a single piezo attached to the tube may generate longitudinal oscillation within the tube. In some embodiments, the tube is arranged to not bend due to the tube's shape structure having a very high bending stiffness due to high moment of inertia of the tube's cross-sectional shape. In such embodiments, vibration may be produced within the tube as the piezo may vibrate with a resonant frequency of the tube, and the cyclical stress waves may force the liquid through the apertures in the aperture plate.

In some embodiments, a plurality of devices may be placed in a linear array. In such an arrangement, one side of the tube may be narrow than another side of the tube such that multiplicity of devices can be stacked together with a minimum space.

In some embodiments, the induced frequency produced by the piezo element may be equal to the natural frequency of the rectangular tube in a longitudinal mode or bending mode.

In some embodiments, the tube may include a rectangular tube having two wide faces such that the area of at least one of the faces is sufficiently wide enough to attach at least one piezoelectric element that is capable of generating a sufficient amplitude.

In some embodiments, the tube may include a trapezoidal cross-sectional shape and have at least one face that is sufficient to attach at least one piezoelectric element that is capable of generating a large amplitude.

In one embodiment the tube may be circular in cross-sectional shape and having one face that is sufficient to attach at least one piezoelectric element that is capable of generating a large amplitude.

In one embodiment, the width of the tube may be between 0.05 mm to 0.1 mm and the length of the tube may be between 1 mm and 45 mm. In some embodiments, it is appreciated that a small device may be preferred for some applications, yet the size may be optimized so as to not require an excessively large resonant frequency. In some embodiments, the aperture plate may be secured to the end of the tube via solder or glue or another affixing method. In such embodiments, the aperture plate may cover the entirety of one end of the tube. In some embodiments, the aperture plate is circular and bent before connecting to an edge of the tube. Additionally, the aperture plates may be flat or domed with the dome shaped extending outward from the end of the tube.

In some applications, the aperture plate may be sized to fit perfectly on the end of the tube. In some implementations, the aperture sizes may be less than about 10 μm. For example, the apertures may be about 5 μm+/−2 μm in some embodiments. In some embodiments, smaller aperture sizes are preferred, but the aperture sizes may be optimized to reduce clogging and the amount of force necessary to generate atomized fluid.

One example use of such a device according to various embodiments includes aerosol generation of scented liquids (such as for an AR/VR application described in an example application). As will be appreciated, the disclosed device also may be used for turning any liquid (e.g., aqueous and non-aqueous) into a mist. In such embodiments, adding the fragrance material may be left out of the aerosolization process (see FIG. 3)

In some embodiment, the device may be used to atomize scented material. For example, scented liquids may be turned into a mist using vibration and micro-pores to allow the scent to permeate in the air in specific quantities.

In other examples, the device may be used to atomize media such as liquid forms of cannabis into aerosol for inhalation. For example, liquid forms of cannabis or cbd oils, waters or other aqueous solutions may be atomized and inhaled by users. Other media that may be used with the device include, but are not limited to, emulsions, solutions, mixtures, and inclusions. In such a case, the generator device may be part of a larger delivery mechanism (e.g., an e-cigarette, vaporizer, or other device) that allows users to inhale atomized liquids or other media types.

In some other applications, the device may be used for dispersing medical liquids (e.g., dispersing certain medicines in an atomized form for inhalation using conventional VMT technology. For instance, VMT devices used in nebulizers could be adapted using some of the embodiments described herein for that purpose.

Other applications of the device may include converting gels to liquids. For example, some theoretic gels have attributes where vibration may turn them from a gel into a liquid, which would allow for atomization through the disclosed device. This may be used to perform gel coatings after the vibration, with the liquid being arranged to coalesce back into a gel thereafter. Another application includes volatile liquid atomization (e.g., with alcohol, ethanol, gasoline, and benzine). For example, in some embodiments, the inventor have recognized the benefit to be able to atomize various less common liquids for reasons like combustion engines. As another application, the device may be used for water humidification.

In some embodiments, the size specification for the device may be relatively small, especially in applications where multiple devices may be used in parallel, such as within a larger device. In other applications (e.g., in an e-cigarette application), the permitted dimension may be limited to a relatively small form factor. Other applications may use a larger form factor, such as a large mist “cannon” that could be used to vaporize large amounts of water or scent or used as part of an engine.

One implementation may include a tube having a rectangular or square in shape. In some conventional piezo elements, this may including using pinching and/or squeezing mechanism to deliver liquids. In some embodiments as disclosed herein, a medium (e.g., a liquid) may be aerosolized via a perpendicular acoustical waves induced by a piezo element. As with the above, there may be multiple ways in which the medium may contact the aperture plate. For example, the medium may be disposed in the housing, a wick may be placed in the tube, and/or a cartridge may be used.

It should be appreciated that there are other applications of this technology and the invention is not limited to the examples provided herein. For example, some embodiments may be used in general entertainment, which could be movies or other experiences. Additionally, some embodiments may be applied to areas such as travel, business, education/training, telepresence, and meditation.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items

What is claimed is:

1. A method of formulating scented nanoemulsions comprising:
providing a first mixture including water and a water surfactant;
providing a second mixture including a fragrance material and a fragrance surfactant;
mixing the first and second mixtures to create a temporary emulsion; and
performing one or more high-energy homogenizations to the temporary emulsion until one or more desired physical properties of a resulting nanoemulsion are obtained.

2. The method according to claim 1, wherein providing the first mixture includes:
mixing the water and water surfactant via high-shear mixing; and
after the step of mixing, applying a sonication process.

3. The method according to claim 1, wherein providing the second mixture includes:
mixing the fragrance and fragrance surfactant via a stir bar; and
after the step of mixing, applying a sonication process.

4. The method according to claim 1, wherein mixing the first and second mixtures to create the temporary emulsion includes high-shear mixing of the first and second mixtures.

5. The method according to claim 1, wherein performing one or more high-energy homogenizations includes performing at least one of a microfluidization, a sonication, or a high-shear mixing.

6. The method according to claim 1, wherein performing one or more high-energy homogenizations includes performing one or more microfluidizations at a pressure of between about 28K and 30K PSI.

7. The method according to claim 6, wherein performing one or more high-energy homogenizations includes, after performing the one or more microfluidizations, performing a sonication process.

8. The method according to claim 5, wherein the one or more high-energy homogenizations are performed via at least one of a microfluidizer, an ultrasonic homogenizer, and a high-shear rotor-stator.

9. The method according to claim 1, further comprising, after the step of performing one or more high-energy homogenizations, measuring the one or more desired physical properties of the resulting nanoemulsion.

10. The method according to claim 9, wherein the one or more desired physical properties include a viscosity, a surface tension, and/or a droplet size.

11. The method according to claim 9, further comprising:
removing excess gas from the resulting nanoemulsion;
adding one or more preservatives; and
adding one or more biocides.

12. The method according to claim 1, further comprising dispersing the resulting nanoemulsion using an aerosol generator.

13. The method according to claim 1, further comprising maintaining the one or more desired physical properties of the resulting nanoemulsion.

14. The method according to claim 13, wherein the one or more desired physical properties of the resulting nanoemulsion include a surface tension, a droplet size, and/or a viscosity.

15. The method according to claim 14, wherein the surface tension is maintained at a level of between 20 mN/m and 72 mN/m.

16. The method according to claim 14, wherein the viscosity is maintained at a level of between about 1 CP and about 24 CP.

17. The method according to claim 14, wherein the droplet size is maintained at a size of not more between about 1 nm and about 5000 nm.

18. The method according to claim 1, wherein the fragrance material includes at least one of oils, waxes, and powders.

19. The method according to claim 1, wherein the fragrance material includes at least one of a lipid-based material and a hydrophobic material.

20. The method according to claim 12, wherein dispersing the resulting nanoemulsion using the aerosol generator includes applying a vibration to the nanoemulsion via a piezoelectric device.

21. The method according to claim 20, wherein the piezoelectric device includes one of a ring-shaped piezo device, a piezoelectric plate, an array of piezo elements.

22. The method according to claim 12, wherein dispersing the resulting nanoemulsion using an aerosol generator includes dispersing one or more scents via an apertured plate.

23. The method according to claim 12, wherein the one or more scents are dispersed via the aerosol generator in response to activities performed or experienced via an XR, AR, or VR device.

24. The method according to claim 12, wherein the nanoemulsion is disposed in a cartridge in the aerosol generator.

25. The method according to claim 1, wherein the fragrance surfactant includes an oil surfactant.

26. The method according to claim 25, wherein the oil surfactant includes span 20 or 80.

27. The method according to claim 1, wherein the water surfactant includes polysorbate 20, 40, 60, or 80.

28. A system for formulating scented nanoemulsions according to the method of claim 1, the system comprising one or more high-energy homogenizers including at least one of a microfluidizer, an ultrasonic homogenizer, and a high-shear rotor stator.

29. A system for formulating scented nanoemulsions according to the method of claim 1, the system comprising:
a first station arranged to perform the step of mixing the first and second mixtures to create the temporary emulsion; and
a second station arranged to perform the one or more high-energy homogenizations to the temporary emulsion.
